(19) United States
(12) Patent Application Publication
(10) Pub. No.: US 2007/0191400 A1
(43) Pub. Date: Aug. 16, 2007

(54) Pharmaceutical compositions comprising anti-inflammatory quinazolinecarboxamide derivatives

(75) Inventors: Paul Gregor, Rehovot (IL); Nicholas Harris, Rehovot (IL); Juraj Koppel, Kosice (SK); Regina Zhuk, Ness Zion (IL)

Correspondence Address: BROWDY AND NEIMARK, P.L.L.C.;624 NINTH STREET, NW, SUITE 300, WASHINGTON, DC, 20001-5303, US

(73) Assignee: Rimonyx Pharmaceuticals Ltd., Ness-Ziona (IL)

(21) Appl. No.: 10/594,081

(22) PCT Filed: Mar. 24, 2005
(86) PCT No.: PCT/IL05/00336

§ 371 Date: Sep. 25, 2006

Related U.S. Application Data
(60) Provisional application No. 60/555,667, filed on Mar. 24, 2004.

(51) Int. Cl. A61K 31/517

(52) U.S. Cl. 514/266.2; 514/266.23; 544/284; 544/287

(57) ABSTRACT

The present invention provides pharmaceutical compositions comprising quinazolinecarboxamide derivative, and certain novel quinazolinecarboxamide derivatives capable of inhibiting heparan sulfate-glycosaminoglycan (HS-GAGs) interactions with L-selectin, and useful in the prevention or treatment of various diseases, disorders and conditions mediated by HS-GAGs, particularly inflammatory and autoimmune diseases, viral diseases, cancer, and amyloid disorders.

### FIELD OF INVENTION

The present invention relates to pharmaceutical compositions comprising quinazolinecarboxamide compounds capable of inhibiting the interactions between L-selectin cell adhesion molecule and glycosaminoglycans (GAGs), particularly heparan sulfate glycosaminoglycans (HS-GAGs), and of inhibiting the interaction between HS-GAGs and cytomegalovirus envelope glycoprotein B. The present invention further relates to methods for the treatment or prevention of diseases or disorders related to cell adhesion and cell migration, particularly for the treatment or prevention of inflammatory and autoimmune diseases and disorders.

### BACKGROUND OF THE INVENTION

The inflammatory response is mediated primarily by leukocytes, neutrophils and lymphocytes, which circulate in the blood and reversibly interact with the vascular endothelium. In response to inflammatory stimuli, the leukocytes adhere tightly to the vascular endothelium, migrate (extravasate) through the vessel wall, and subsequently move along a chemotactic gradient toward the inflammatory stimulus. The interaction of leukocytes with vascular endothelial cells is thus an essential initial step in the inflammatory response. Selectins play a key role in inflammation, as they are responsible for the initial attachment of blood borne leukocytes to the vasculature. Preventing selectin-mediated cell adhesion can ameliorate or circumvent the deleterious consequences of inflammation. Therefore, selectins are the prime target for the therapy of cell-adhesion disorders, specifically for treatment of inflammation.

Selectins regulate neutrophil and lymphocyte adhesion to and entry into lymphoid tissues and sites of inflammation. The three known selectins are E-selectin (formerly known as ELAM.1), P-selectin (formerly known as PADGEM, GMP-140, or CD61) and L-selectin (formerly known as mLHR, Leu8, TQ-1, gp90, MEL, Lam-1, or Lecam-1) (Lasky, L. Annu. Rev. Biochem. 64:113, 1995). Each selectin is regulated differently, and participates in a different manner in the process of inflammation or immunity. The lectin domains of each selectin are critical to the adhesive functions of the proteins. The selectins are responsible for leukocyte capture from the blood stream and mediate their intermittent attachment with consequent leukocyte “rolling” along the endothelial cell surface. This capture allows the cascade of secondary, tighter cell-adhesive events to take place. In inflammatory disorders it may be L-selectin that plays the most significant role (L. Lasky, ibid). Monoclonal antibody SMART is an L-selectin blocking antibody that is being used in clinical trials for trauma associated with multiple organ failure (this condition is believed to be due in part to infiltration of inflammatory cells). The anti-L-selectin antibody is presumed to provide its therapeutic effect by preventing neutrophil adhesion to endothelium and it is active in vivo in a primate model of severe trauma (Schlag G et al, Critical Care Medicine 1999, 27, 1900-1907). It is believed that this monoclonal antibody will be also useful in the treatment of adult respiratory distress syndrome and myocardial infarction.

Glycosaminoglycans (also referred to herein as “GAG” or “GAGs”) are naturally-occurring carbohydrate-based molecules implicated in the regulation of a number of cellular processes, including blood coagulation, angiogenesis, tumor growth, and smooth muscle cell proliferation, most likely by interaction with effector molecules. GAGs are often, but not always, found covalently bound to protein cores in structures called proteoglycans. Proteoglycan structures are abundant on cell surfaces and are associated with the extracellular matrix around cells. GAGs consist of repeating disaccharide units. For example, heparan sulfate glycosaminoglycans (also referred to herein as “HS-GAGs”) consist of repeating disaccharide units of D-glucuronic acid and N-acetyl- or N-sulfo-D-glucosamine. The high molecular diversity of HS-GAGs is due to their unique sulfation pattern (Sasisekharan, R. and Venkataraman, G., Current Opinion in Chem. Biol., 4, 626-631, 2000). One of the most thoroughly studied HS-GAGs is the widely used anticoagulant heparin. Heparin is a highly sulfated form of heparan sulfate found in mast cells. Many important regulatory proteins including cytokines, growth factors, enzymes, and cell adhesion molecules bind tightly to heparin. Although interactions of proteins with GAGs such as heparin and heparan sulfate are of great biological importance, the structural requirements for protein-GAG binding have not been well characterized. Ionic interactions are important in promoting protein-GAG binding and the spacing of the charged residues may determine protein-GAG affinity and specificity.

The HS-GAG paradigm provides new approaches and strategies for therapeutic intervention at the cell-tissue-organ interface. For example, identification of specific HS-GAG sequences that affect particular biological processes will enable the development of novel molecular therapeutics based on polysaccharide sequence. Synthetic HS-GAGs, or molecular mimics of HS-GAG sequences, may provide new approaches for combating health problems such as bacterial and viral infections, atherosclerosis, cancer, and Alzheimer's disease.

Selectins mediate their adhesive functions via lectin domains that bind to carbohydrate ligands. Emerging evidence indicates that GAGs, and in particular HS-GAGs, are carbohydrate receptors with which the selectins interact (Nelson R. M., et al., Blood 82, 3253-3258, 1993). Consistent with this observation, heparin, HS-GAG and heparin-derived oligosaccharides block L-selectin-dependent adhesion directly and short sulfated heparin-derived tetrasaccharides reduced binding of neutrophils to COS cells expressing P-selectin (Nelson R. M., ibid). The multivalent nature of HS may be an important factor in binding L-selectin under flow conditions.

As the interactions between GAGs and selectins play an important role in cell-matrix and cell-cell adhesion and the latter are processes involved in certain diseases and inflammatory disorders, the modulation of these interactions may have therapeutic implications.

Xie X et al (J. Biol. Chem. 275, 34818-25, 2000) described inhibition of L-30 and P-selectin mediated cell adhesion by sulfated saccharides, including carboxyl-reduced and sulfated heparin. While these molecules have been useful to show the utility of selectin blockers for treating inflammation, each has significant drawbacks as a therapeutic, including short in vivo half-life, high cost, potential immunogenicity, and other possible side effects. A further limitation of these approaches is lack of efficient means to improve the pharmacological properties of these molecules.

A growing body of evidence points to the role of cell surface GAGs as the initial receptor in viral infection. Specifically, viruses such as Herpes Simplex Virus (HSV), Dengue Virus, Respiratory Syncytial Virus, Varicella-zoster virus, Cytomegalovirus (Boyle K A and Compton T 1998, J Virol. 72, 1826-1833), Sindbis Virus, Adeno-associated Virus, Vaccinia Virus, Foot-and-mouth Disease Virus and HIV-1, all employ HS-GAGs for their initial step of infection.

There is still an unmet need for non-peptidic, small synthetic compounds, which are capable of modulating the functions of GAGs and the interactions between GAGs and GAG effector protein molecules.

Synthesis of certain quinazoline derivatives has been described by Ivatchtchenko, A. V., Kovalenko, S. M. and Drushlyak, O. G., J. Comb. Chem. 5, 775-788, 2003. However, the manuscript does not describe or suggest that these derivatives of quinazolines have any beneficial pharmaceutical activities.

SciFinder Scholar database, release 2005, lists 888 derivatives (as of Mar. 10, 2005) of quinazolinecarboxamide, but no utility is attributed to any of these compounds and no chemical synthesis data are described.

Chemical Diversity Labs Inc. (San Diego, Calif.), a supplier of chemical compounds, released a database of about 500,000 compound structures (January 2005), which lists 623 derivatives of quinazolinecarboxamide, but no utility or chemical synthesis data is described.

Enamine (Kiev, Ukraine), a supplier of chemical compounds, released a database (January 2005; 355,966 compound structures), which lists 11 derivatives of quinazolinecarboxamide, but no utility or chemical synthesis data is described.

As far as known by Applicant, the background art has not taught or suggested that the derivatives of quinazolinecarboxamide described herein in the present application have any biological or pharmaceutical activity.

### SUMMARY OF THE INVENTION

It is an object of some aspects of the present invention to provide pharmaceutical compositions comprising small organic compounds for medical and diagnostic use, wherein the small organic compounds are inhibitors of the interactions between cell adhesion molecules, specifically L-selectin, with glycosaminoglycans (GAGs), specifically heparan sulfate glycosaminoglycans (HS-GAGs). Accordingly, these compositions are useful as inhibitors of cell-cell interactions mediated by L-selectin, particularly leukocyte adhesion, migration and infiltration. In addition, said compositions inhibit HS-GAG interaction with CMV envelope glycoprotein B and may be therefore useful as inhibitors CMV infection.

According to one aspect, the present invention provides a pharmaceutical composition comprising a pharmaceutically acceptable diluent or carrier and a compound of the general formula I:

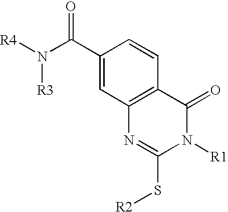

wherein:

R₁ is optionally substituted hydrocarbyl or heterocyclyl;

R₂ is H, (C₁-C₁₂)alkyl, (C₆-C₁₄)aryl-CH₂-, heteroaryl-CH₂-, alkylcarbonyl-CH ₂-, (C₆-C₁₄)arylcarbonyl-CH₂-, or heteroarylcarbonyl-CH₂-;

R₃ and R4 each is selected from the group consisting of hydrogen, C₁-C₆ alkyl, (C₁-C₆)alkoxy(C₁-C₆)alkyl, and C₁-C₆ alkyl substituted by a group containing a basic nitrogen atom or by a 5-7 membered heterocyclic ring containing one or two heteroatoms, one of them being a basic nitrogen atom, or R₃ and R4 together with the nitrogen atom to which they are attached form a 5-7 membered saturated heterocyclic ring containing one or two basic nitrogen atoms, optionally substituted on the additional nitrogen atom;

and pharmaceutically acceptable salts thereof.

According to one embodiment, the compounds of formula I of the pharmaceutical compositions of the present invention inhibit the interactions of HS-GAGs with selecting, specifically L-selectin.

According to another embodiment, the compounds of formula I of the pharmaceutical compositions of the present invention bind directly to GAGs, specifically HS-GAG.

According to yet another embodiment, the present invention provides pharmaceutical composition comprising compounds of general formula I capable of inhibiting the interactions between HS-GAGs and cytomegalovirus envelope glycoprotein B.

According to a further embodiment, the compounds of formula I of the pharmaceutical compositions of the present invention inhibit leukocyte and neutrophil infiltration in vivo.

According to still another aspect, the present invention provides a method for the treatment or prevention of diseases and disorders related to cell adhesion and cell migration mediated by GAG-L-selectin interaction, comprising the step of administering to a subject in need thereof a therapeutically effective amount of a pharmaceutical composition comprising a compound of the general formula I.

According to yet another aspect, the invention provides a method for treatment or prevention of diseases or disorders mediated by GAGs, wherein the GAGs are selected from the group consisting of heparan sulfate (HS-GAG), heparin, chondroitin sulfate, dermatan sulfate, keratan sulfate, and derivatives and fragments thereof. According to a currently preferred embodiment, the GAG is HS-GAG.

According to another embodiment, the disease or disorder mediated by GAG-L-selectin interaction is selected from inflammatory processes or disorders, autoimmune processes or diseases, platelet-mediated pathologies, cancer, tumor metastasis, viral diseases, coagulation disorders, atherosclerosis, amyloid disorders, and kidney diseases.

According to another embodiment, the inflammatory processes or disorders mediated by GAG-L-selectin interaction are exemplified by, but not restricted to, septic shock, post-ischemic leukocyte-mediated tissue damage, frost-bite injury or shock, acute leukocyte-mediated lung injury, acute pancreatitis, nephritis, asthma, traumatic shock, stroke, traumatic brain injury, nephritis, acute and chronic inflammation, including atopic dermatitis, psoriasis, uveitis, retinitis, and inflammatory bowel disease.

According to yet another embodiment, the autoimmune diseases mediated by GAG-L-selectin interactions are exemplified by, but not restricted to, rheumatoid arthritis and multiple sclerosis.

According to yet further embodiment, the invention provides a method for the treatment or prevention of diseases and disorders mediated by GAGs, specifically HS-GAG. The diseases and disorders mediated by HS-GAGs are selected from the group consisting of amyloid disorders such as Alzheimer's disease and type II diabetes; viral diseases such as hepatitis C and B, influenza, rhinovirus infections, cytomegalovirus infections, AIDS, and respiratory syncytial virus infections; bacterial infections and malaria; kidney diseases; cancer; and coagulation disorders.

According to another aspect, the present invention relates to the use of a compound of the general formula I for the preparation of a pharmaceutical composition.

According to yet another aspect, the present invention provides certain novel compounds of the general formula I, namely, the compounds 2-[[(6-nitro-4H-1,3-benzodioxin-8-yl)methyl]thio]-3-(2-propenyl)-3,4-dihydro-4-oxo-N-[3-(4-morpholinyl)propyl ]-7-quinazolinecarboxamide (Compound No. 2010), 2-[[(5-acetyl-2-methoxyphenyl)methyl)thio]-3-(phenylmethyl)-3,4-dihydro-4-oxo-N-[3-(1H-imidazol-1-yl)propyl]-7-quinazolinecarboxamide (Compound No. 2011), and 2-[[(5-acetyl-2-methoxyphenyl)methyl]thio]-3-(phenylmethyl)-3,4-dihydro-4-oxo-N -(1-ethyl-piperidin-4-yl)-7-quinazolinecarboxamide (Compound No. 2012).

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 shows the anti-inflammatory properties of Compound No. 2 administered orally at 25 mg/kg in a model of Delayed Type Hypersensitivity (DTH). The y axis represents difference in ear thickness in mm. There were 12 mice per group and the data were statistically significant as determined by Student's t-test with p>0.05.

FIG. 2 shows the anti-inflammatory properties of Compound No. 1 administered intraperitoneally at 10 mg/kg in a model of mouse peritonitis. The y axis displays counts of neutrophil per volume unit. There were 15 mice per group and the data were statistically significant as determined by Student's t-test with p>0.001.

FIG. 3 shows the anti-inflammatory properties of Compound No. 1 administered orally at 1 mg/kg in Paw Edema, 24 hours after induction. The y axis represents difference in paw thickness in mm. There were 12 mice per group and the data were statistically significant as determined by Student's t-test with p>0.001.

FIG. 4 shows the anti-inflammatory properties of Compound No. 9 administered orally at 50 mg/kg in a model of paw edema. The measurements were taken after 24 hours. The y axis represents difference in paw thickness in mm. There were 12 mice per group and the data were statistically significant as determined by Student's t-test with p>0.001.

### DETAILED DESCRIPTION OF THE INVENTION

The present invention provides pharmaceutical compositions comprising a compound of the general formula I herein, wherein

R₁ is optionally substituted hydrocarbyl, or heterocyclyl;

R₂ is H, (C₁-C₁₂)alkyl, (C₆-C₁₄)aryl-CH₂-, heteroaryl-CH₂-, alkylcarbonyl-CH ₂-, (C₆-C₁₄)arylcarbonyl-CH₂-, or heteroarylcarbonyl-CH₂-;

R₃ and R4 each is selected from the group consisting of hydrogen, C₁-C₆ alkyl, (C₁-C₆)alkoxy(C₁-C₆)alkyl, and C₁-C₆ alkyl substituted by a group containing a basic nitrogen atom or by a 5-7 membered heterocyclic ring containing one or two heteroatoms, one of them being a basic nitrogen atom, or R₃ and R₄ together with the nitrogen atom to which they are attached form a 5-7 membered saturated heterocyclic ring containing one or two basic nitrogen atoms, optionally substituted on the additional nitrogen atom;

and pharmaceutically acceptable salts thereof.

As defined herein, the term “hydrocarbyl” refers to a radical containing only carbon and hydrogen atoms that may be saturated or unsaturated, linear or branched, cyclic or acyclic, or aromatic, and include C₁-C₁₂ alkyl, C₂-C₁₂ alkenyl, C₂-C₁₂ alkynyl, C₃-C₁₀ cycloalkyl, C₃-C₁₀ cycloalkenyl, C₆-C₁₄ aryl, (C₁-C ₆)alkyl(C₆-C₁₄)aryl, and (C₆-C₁₄) aryl(C₁-C₁₂)alkyl.

The term “C₁-C₁₂ alkyl” typically refers to a straight or branched alkyl radical having 1-12 carbon atoms and includes, for example, methyl, ethyl, n-propyl, isopropyl, n-butyl, isobutyl, tert-butyl, n-pentyl, isopentyl, 2,2-dimethylpropyl, n-hexyl, n-heptyl, n-octyl, n-nonyl, n-decyl, and the like.

The term “C₂-C₁₂ alkenyl” refers to a straight or branched hydrocarbon radical having 2-12 carbon atoms and one or more double bonds, and includes for example vinyl, allyl, but-3-en-1-yl, pent-4-en-1-yl, hex-5-en-1-yl, and the like.

The term “C₂-C₁₂ alkynyl” refers to a straight or branched hydrocarbon radical having 2-12 carbon atoms and one or more triple bonds, and includes for example ethynyl, propynyl, butynyl, octynyl, and the like.

The term “C₃-C₁₀ cycloalkyl” refers to a saturated cyclic hydrocarbon radical of 3-10 carbon atoms such as cyclopropyl, cyclobutyl, cyclohexyl, cycloheptyl, and the like, and the term “C₃-C₁₀ cycloalkenyl” to such a saturated ring such as cyclobutenyl, cyclopentenyl, cyclohexenyl, and the like.

The term “C₆-C₁₄ aryl” refers to an aromatic carbocyclic group having 6 to 14 carbon atoms consisting of a single ring or multiple condensed rings such as phenyl, naphthyl, carbazolyl and phenanthryl.

The term “(C₆-C₁₄) aryl(C₁-C₁₂)alkyl” refers to an aralkyl radical such as benzyl, phenethyl, phenylpropyl, phenylhexyl, naphthylmethyl, naphthylethyl, and the like.

The term “heterocyclyl” refers to a radical derived from a mono- or poly-cyclic ring containing one to three heteroatoms selected from the group consisting of N, O and S, with or without unsaturation or aromatic character. The term “heteroaryl” refers to such a mono- or poly-cyclic ring having aromatic character. Non-limiting examples of non-aromatic heterocyclyl include dihydrofuryl, tetrahydrofuryl, dihydrothienyl, pyrrolydinyl, pyrrolynyl, dihydropyridyl, piperidinyl, piperazinyl, morpholino, 1,3-dioxanyl, and the like. A polycyclic ring may have the rings fused, as in quinoline or benzofuran, or unfused as in 4-phenylpyridine. Non-limiting examples of heteroaryl include pyrrolyl, furyl, thienyl, pyrazolyl, imidazolyl, oxazolyl, isoxazolyl thiazolyl, isothiazolyl, pyridyl, 1,3-benzodioxinyl, pyrazinyl, pyrimidinyl, 1,3,4-triazinyl, 1,2,3-triazinyl, 1,3,5-triazinyl, thiazinyl, quinolinyl, isoquinolinyl, benzofuryl, isobenzofuryl, indolyl, imidazo[1,2-a]pyridyl, pyrido[1,2-a]pyrimidinyl, benzimidazolyl, benzthiazolyl, benzoxazolyl and the like. It is to be understood that when a polycyclic heteroaromatic ring is substituted, the substitutions may be in any of the carbocyclic and/or heterocyclic rings.

The hydrocarbyl, particularly any alkyl and aryl, and any heteroaryl or heterocyclyl radical may be substituted by one or more radicals including, but not limited to, halogen, hydroxy, C₁-C₁₀ alkyl, C₂-C₁₀ alkenyl, C₂-C₁₀ alkynyl, C₇-C₁₂ aralkyl, C₆-C₁₀ aryl, C₇-C₁₂ alkaryl, C₁-C₁₀ alkoxy, C₆-C₁₀ aryloxy, C₁-C₁₀ alkylthio, C₆-C₁₀ arylthio, C₆-C₁₀ arylamino, C₃-C₁₀ cycloalkyl, C₃-C₁₀ cycloalkenyl, amino, C₁-C₁₀ alkylamino, di(C₁-C₁₀)-alkylamino, C₂-C₁₂ alkoxyalkyl, C₂-C₁₂ alkylthioalkyl, C₁-C₁₀ alkylsulfinyl, C₁-C₁₀ alkylsulfonyl, C₆-C₁₀ arylsulfonyl, hydroxy(C₁-C₁₀)alkyl, (C₆-C₁₀)aryloxy(C₁-C₁₀)alkyl, (C₁-C₁₀)alkoxycarbonyl, (C₆-C₁₀)aryloxycarbonyl, C₂-C₁₁, alkanoyl, (C₇-C₁₁)aroyl, fluoro(C₁-C₁₀)alkyl, oxo, nitro, nitro(C₁-C₁₀)alkyl, cyano, cyano(C₁-C₁₀)alkyl, aminocarbonyl, (C₁-C₁₀)alkylaminocarbonyl, di(C₁-C₁₀)-alkylaminocarbonyl, aminocarbonyl(C₁-C₁₀)alkyl, aminocarbonyl(C₆-C₁₀)aryl, aminosulfonyl, (C₁-C₁₀)alkylaminosulfonyl, di(C₁-C₁₀)-alkylaminosulfonyl, amidino, carboxy, sulfo, heterocyclyl, and —(CH₂)ₘ-Z-(C₁-C₁₀ alkyl), where m is 1 to 8 and Z is oxygen or sulfur.

The term “halogen” refers to fluoro, chloro, bromo or iodo. A “haloalkyl” group refers to an alkyl group as defined above, which is substituted by one or more halogen atoms.

The term “(C₁-C₁₀) alkoxy” refers to the group (C₁-C₁₀) alkyl-O-, wherein (C₁-C₁₀) alkyl is as defined above. Examples of alkoxy are methoxy, ethoxy, butoxy, hexoxy, and the like.

As defined above, R₃ and R₄ each may be a C₁-C₆ alkyl substituted by a group containing a basic nitrogen atom or by a 5-7 membered heterocyclic ring containing one or two heteroatoms, one of them being a basic nitrogen atom, or R₃ and R₄ together with the nitrogen atom to which they are attached form a 5-7 membered saturated heterocyclic ring containing one or two basic nitrogen atoms, optionally substituted on the additional nitrogen atom.

The term “a group containing a basic nitrogen atom” refers to groups including, but not limited to, an amino group —NR₅R₆, an ammonium group —N N⁺(R₅R₆R₇), a hydrazine group —NR₅—NR₆R₇, a hydrazonium group —NR₅—N ⁺(R₆R₇R₈), an ammoniumoxy group —O—N⁺(R₅R₆), an imine group —C═NR₅R₆, an iminium group —C═N³⁰ (R₅R₆R₇), a guanidine group —NR₅—C(═NH)—NR₆R₇, and a guanidinium group —NR₅—C(═NH)—N⁺(R₆R₇R₈), wherein each of R₅, R₆, R₇ and R₈ is H, or optionally substituted C₁-C₁₀ alkyl or C₆-C₁₀ aryl, as defined hereinabove.

The term “5-7 membered heterocyclic ring containing one or two heteroatoms, one of them being a basic nitrogen atom” refers to both saturated, unsaturated and aromatic rings containing one or two nitrogen atoms such as pyrrolidine, pyrroline, pyrrol, imidazolidine, imidazoline, imidazole, piperidine, dihydropyridine, tetrahydropyridine, pyridine, 1,2-pyrazine, tetrahydropyrimidine, dihydropyrimidine, pyrimidine, 1,4-pyrazine, 1,4-tetrahydropyrazine, 1,4-dihydropyrazine, piperazine, diazepine, and the like; or containing one nitrogen atom and one oxygen atom such as oxazolidine, oxazoline, oxazole, morpholino, 1,4-dihydrooxazine, 1,4-oxazine, and the like; or containing one nitrogen atom and one sulfur atom such as thiazolidine, thiazoline, thiaazole, thiomorpholino, 1,4-dihydrothiazine, 1,4-thiazine and the like.

When R₃ and R₄ together with the nitrogen atom to which they are attached form a 5-7 membered saturated heterocyclic ring containing one or two basic nitrogen atoms, optionally substituted on the additional nitrogen atom, the term “5-7 membered saturated heterocyclic ring containing one or two basic nitrogen atoms” includes, without limitation, the rings pyrrolidine, imidazolidine, piperidine, piperazine, and the like. The substituent at the additional nitrogen atom may be C₁-C₆ alkyl, optionally substituted by halo, hydroxy, C₁-C₆ alkoxy or C₆-C₁₀ aryl, or C₂-C₇ alkoxycarbonyl.

It is to be understood that the term “substituted”, as used herein, means that any one or more hydrogen on the designated atom is replaced with a selection from the indicated groups, provided that the designated atom's normal valency is not exceeded, and that the substitution results in a stable compound. Combinations of substituents are permissible only if such combinations result in stable compounds. By “stable compound” or “stable structure” it is meant herein a compound that is sufficiently robust to survive isolation to a useful degree of purity from a reaction mixture, and formulation into an efficacious therapeutic agent.

As contemplated herein, the present invention further encompasses isomers, pharmaceutically acceptable salts and hydrates of the compounds defined by the present invention.

The term “isomer” includes, but is not limited to, optical isomers, structural isomers, conformational isomers, and the like. Thus, the present invention encompasses various optical isomers of the compounds of the present invention. It will be appreciated by those skilled in the art that the compounds of the present invention contain at least one chiral center. Accordingly, these compounds exist in, and are isolated in, optically active or racemic forms. Unless otherwise indicated, all chiral, diastereomeric and racemic forms of the compounds described in the present invention are encompassed by the present invention. The compounds may also have asymmetric centers. Many geometric isomers of olefins, C- and N-double bonds and the like can also be present in the compounds described herein, and all such stable isomers are contemplated in the present invention. It will be appreciated that compounds of the present invention that contain asymmetrically substituted carbon atoms may be isolated in optically active or racemic forms. It is well known in the art how to prepare optically active forms, for example, by resolution of the racemic form by recrystallization techniques, by synthesis from optically active starting materials, by chiral synthesis, or by chromatographic separation using a chiral stationary phase. All chiral, diastereomeric, racemic forms and all geometric isomeric forms of a structure are intended, unless the specific stereochemistry or isomer form is specifically indicated.

Some compounds may also exhibit polymorphism. It is to be understood that the present invention encompasses any racemic, optically active, polymorphic, or stereroisomeric form, or mixtures thereof. In one embodiment, the compounds are the pure (R)-isomers. In another embodiment, the compounds are the pure (S)-isomers. In another embodiment, the compounds are a mixture of the (R) and the (S) isomers. In another embodiment, the compounds are a racemic mixture comprising an equal amount of the (R) and the (S) isomers.

In addition, this invention further includes hydrates of the compounds described herein. The term “hydrate” includes but is not limited to hemihydrate, monohydrate, dihydrate, trihydrate, and the like.

The compounds of the present invention can also be in the form of prodrugs. Prodrugs are considered to be any covalently bonded carriers that release the active parent drug according to Formula I in vivo, when such prodrug is administered to a mammalian subject. Prodrugs of the compounds of Formula I are prepared by modifying functional groups present in the compounds in such a way that the modifications are cleaved, either in routine manipulation or in vivo, to provide the parent compound of Formula I. Prodrugs include compounds of Formula I wherein hydroxyl, amino, sulfhydryl, or carboxyl groups are bonded to any group that, when administered to a mammalian subject, are cleaved to form a free hydroxyl, amino, sulfhydryl, or carboxyl group, respectively. Examples of prodrugs include, but are not limited to, acetate, formate, and benzoate derivatives of alcohol and amine functional groups in the compounds of Formula I, and the like.

According to one embodiment, R₁ is C₁-C₁₂ alkyl, preferably C₁-C₆ alkyl, more preferably pentyl, or R₁ is C₂-C₁₂ alkenyl, preferably C₂-C₆ alkenyl, more preferably allyl, or R₁ is C₆-C₁₄ aryl, preferably C₆-C₁₀ aryl, more preferably phenyl, optionally substituted by halogen, preferably by fluor.

According to another embodiment, R₁ is C₁-C₁₂ alkyl, preferably C₁-C₆ alkyl, more preferably methyl, substituted aryl, preferably phenyl, or by heterocyclyl, preferably by furyl or tetrahydrofuryl.

According to one embodiment, R₂ is hydrogen or C₁-C₁₂ alkyl. According to another embodiment, R₂ is methyl (—CH₂—) substituted by C₆-C₁₄ aryl, preferably C₆-C₁₀ aryl, more preferably phenyl, optionally substituted by C₁-C₁₂ alkyl, preferably C₁-C₆ alkyl, more preferably methyl, or by halogen, preferably chloro or fluor.

According to another embodiment, R₂ is methyl (—CH₂—) substituted by heterocyclyl, preferably a mono- or di-heterocyclic ring containing one or more nitrogen atoms, more preferably, 4-pyridyl or 4-oxo-4H-pyrido[1,2-a]pyrimidin-2-yl.

According to yet another embodiment, R₂ is methyl (—CH₂—) substituted by alkanoyl or by (C₇-C₁₁)aroyl, preferably phenylcarbonyl, wherein the phenyl is optionally substituted by fluor or chloro, or R₂ is methyl (—CH₂—) substituted by heteroaryl.

According to one embodiment, R₃ is hydrogen and R₄ is C₁-C₆ alkyl substituted by alkoxy, preferably 2-methoxyethyl, or R₄ is C₁-C₆ alkyl, preferably propyl, substituted by a 5-7 membered heterocyclic ring containing one or two heteroatoms, one of them being a basic nitrogen atom, preferably 4-morpholinyl, piperidin-1-yl, piperidin-4-yl or imidazolyl.

According to another embodiment, R₃ and R₄ together with the N atom to which they are attached form a piperazine ring optionally substituted at the additional N atom by C₁-C₆ alkyl, preferably methyl, or by C₂-C₇ alkoxycarbonyl, preferably ethoxycarbonyl.

According to certain preferred embodiment, the present invention provides compositions comprising one or more of the following compounds of formula I:

2-[[(4-chlorophenyl)methyl]thio]-3-(4-fluorophenyl)-3,4-dihydro-4-oxo-N-[3-(4-morpholinyl)propyl]-7-quinazolinecarboxamide

2-[[(4-methylphenyl)methyl]thio]-3-(4-fluorophenyl)-3,4-dihydro-4-oxo-N-[3-(4-morpholinyl)propyl]-7-quinazolinecarboxamide

2-[[(3-fluorophenyl)methyl]thio]-3-(4-fluorophenyl)-3,4-dihydro-4-oxo-N-[3-(4-morpholinyl)propyl]-7-quinazolinecarboxamide

2-[(2-oxo-2-phenylethyl)thio]-3-(4-fluorophenyl)-3,4-dihydro-4-oxo-N-[3-(4-morpholinyl)propyl]-7-quinazolinecarboxamide

2-[[(4-oxo-4H-pyrido [1,2-a]pyrimidin-2-yl)methyl]thio]-3-(2-furanylmethyl) -3,4-dihydro-4-oxo-N-(2-methoxyethyl)-7-quinazolinecarboxamide

2-[(2-oxo-2-phenylethyl)thio]-3-[(tetrahydro-2-furanyl)methyl]-3,4-dihydro-4-oxo -N-[3-(1-piperidinyl)propyl]-7-quinazolinecarboxamide

4-[[3,4-dihydro-4-oxo-3-pentyl-2-[(4-pyridinylmethyl)thio]-7-quinazolinyl]carbonyl]-1-piperazinecarboxylic acid ethyl ester

2-[[2-[[(3-chlorophenyl)methyl]thio]-3-pentyl-3,4-dihydro-4-oxo-N-(4-methylpiperazin -7-quinazolinecarboxamide

2-[[2-oxo-2-(4-fluorophenyl)ethyl]thio]-3-[(tetrahydro-2-furanyl)methyl]-3,4-dihydro-4-oxo-N-[3-(4-morpholinyl)propyl]-7-quinazolinecarboxamide.

2-mercapto-3-(2-methylpropyl-3)-3,4-dihydro-4-oxo-N-[3-(1N-imidazol-1-yl)propyl]-7-quinazolinecarboxamide [Compound 9].

Some of the compounds of the general formula I disclosed herein are novel compounds and are claimed in the present application as such. These compounds include:

2-[[(6-nitro-4H-1,3-benzodioxin-8-yl)methyl]thio]-3-(2-propenyl)-3,4-dihydro -4-oxo-N-[3-(4-morpholinyl)propyl]-7-quinazolinecarboxamide (Compound No. 2010).

2-[[(5-acetyl-2-methoxyphenyl)methyl)thio]-3-(phenylmethyl)-3,4-dihydro-4-oxo-N-[3-(1H-imidazol-1-yl)propyl]-7-quinazolinecarboxamide (Compound No. 2011).

2-[[(5-acetyl-2-methoxyphenyl)methyl]thio]-3-(phenylmethyl)-3,4-dihydro-4-oxo-N-(1-ethyl-piperidin-4-yl)-7-quinazolinecarboxamide (Compound No. 2012).

The synthesis of the three novel compounds is described herein below in Examples 1-4.

In accordance with the present invention and as used herein when referring to the biological activity of the compounds of general formula I, the following terms are defined with the following meanings, unless explicitly stated otherwise.

The term “GAG” refers to glycosaminoglycans, including heparan sulfate (HS-GAG), heparin, chondroitin sulfate, dermatan sulfate and keratan sulfate. It includes the GAG chains of proteoglycans such as heparan sulfate proteoglycan or chondroitin sulfate proteoglycan. It includes fragments of GAG produced chemically or enzymatically. It also includes derivatives of GAG, which may be produced by chemical or enzymatic means as known in the art. GAG may be free or attached to a linker, support, cell or a protein. GAGs may be crude or purified from organs, tissues or cells.

The term “HS-GAG” refers to heparan sulfate glycosaminoglycan. It includes fragments of heparan sulfate such as those that may be produced chemically, enzymatically or during purification. It includes the HS-GAG chains of proteoglycans such as heparan sulfate proteoglycans. HS-GAG may be free or attached to a linker, support, cell or protein, or otherwise chemically or enzymatically modified. HS-GAGs may be crude or purified from organs, tissues or cells.

“HS-PG” refers to heparan sulfate proteoglycans.

“Heparin” is polysulfated polysaccharide, with no protein associated with it. As used herein, heparin refers to heparin prepared from different organs or species such as from porcine intestinal mucosa. The invention encompasses heparins with various molecular weights including low molecular weight heparins (LMWHs), such as commercially available Fraxiparin, and other heparin derivatives, prepared or modified by chemical or enzymatic reactions as known in the art.

“GAG Derivatives” consist of products derived from GAGs, made by one or more chemical or enzymatic modifications. The modifications are designed to modify the activity of relevant groups of the molecules.

“Oligosaccharide fragments” or “GAG-Derived Oligosaccharides” are products derived from GAGs by controlled cleavage and preferably purified after cleavage.

The terms “L-selectin/IgG” and “P-selectin/IgG” refer to a selectin chimera molecule, in which an N-terminal portion of the selectin comprising the binding domain is fused to an IgG Fc region (Aruffo et al., Cell 67:35, 1991; and Foxall et al. J. Cell Biol. 117:895, 1992).

The term “Inhibitor Compound” refers to a small organic compound that inhibits, modulates or reverses the function of a GAG. For instance, the inhibitor Compound may inhibit interaction (binding) between two molecules: (1) a GAG, exemplified by, but not restricted to, heparin, or HS-GAG and (2) L-selectin.

The terms “inflammation”, “inflammatory diseases”, “inflammatory condition” or “inflammatory process” are meant as physiological or pathological conditions, which are accompanied by an inflammatory response. Such conditions include, but are not limited to, sepsis, ischemia-reperfusion injury, Crohn's disease, rheumatoid arthritis, multiple sclerosis, cardiomyopathic disease, colitis, infectious meningitis, encephalitis, acute respiratory distress syndrome, organ/tissue transplant rejection (such as skin, kidney, heart, lung, liver, bone marrow, cornea, pancreas, small bowel), dermatitis, stroke, traumatic brain injury, psoriasis and lupus.

The term “treatment” or “treating” is intended to include the administration of the compound of the invention to a subject for purposes which may include prophylaxis, amelioration, prevention or cure of disorders mediated by cell adhesion or cell migration events, specifically selectin adhesion events, more specifically L-selectin and P-selectin-mediated adhesion events. Such treatment need not necessarily completely ameliorate the inflammatory response or other responses related to the specific disorder. Further, such treatment may be used as sole treatment or in conjunction with other traditional treatments for reducing the deleterious effects of the disease, disorder or condition as known to those of skill in the art.

The methods of the invention may be provided as a “preventive” treatment before detection of, for example, an inflammatory state, so as to prevent the disorder from developing in patients at high risk for the same, such as, for example, transplant patients.

The term “cancer” refers to various cancer-associated conditions including metastasis, tumor growth, and angiogenesis. According to the invention, cancer is exemplified by leukemias.

As used through this specification and the appended claims, the singular forms “a”, “an” and “the” include the plural unless the context clearly dictates otherwise. Thus, for example, reference to “a compound” includes mixtures of such compounds, reference to “a P-selectin”, or “an L-selectin” includes reference to respective mixtures of such molecules, reference to “the formulation” or “the method” includes one or more formulations, methods and/or steps of the type described herein and/or which will become apparent to those persons skilled in the art upon reading this disclosure.

The present invention relates to pharmaceutical compositions comprising as an active ingredient at least one compound of the general formula I capable of inhibiting the interactions of glycosaminoglycans (GAGs) with selecting.

According to a preferred embodiment, the compounds of the present invention inhibit the interactions of HS-GAGs with L-selectin (see Example 6 and Table 1 herein below).

According to yet another embodiment, the compounds of the present invention bind directly to GAGs, specifically HS-GAG. The compounds of the invention can thus be employed for treatment or prevention of diseases and disorders mediated by GAGs.

According to yet another embodiment, the present invention provides a method for inhibiting cell adhesion and cell migration in vitro comprising the step of exposing the cells to at least one compound according to formula I in an amount sufficient to inhibit GAG to L-selectin interactions (see Example 6 herein below).

The inhibitory effect of the compounds of the present invention can be evaluated by several methods in vitro. One assay for measuring GAG-L-selectin binding, exemplified hereinbelow, detects the binding of L-selectin/IgG to immobilized heparin. Another assay utilizes immobilized L-selectin, or L-selectin fused to protein domains other, than IgG. The amount of bound L-selectin is determined by an ELISA assay using, for example, a monoclonal antibody raised against L-selectin, which is conjugated to horseradish peroxidase.

The biological activity of the compounds according to formula I of the present invention may be assayed in a variety of systems. For example, a compound can be immobilized on a solid surface and adhesion of cells expressing HS-GAGs can be measured. The test compounds can also be tested for the ability to competitively inhibit binding between HS-GAGs and other proteins binding to HS-GAGs such as other cell adhesion molecules, cytokines, or viral proteins. Many assay formats employ radioactive or non-radioactive labeled assay components. The labeling systems can be in a variety of forms. The label may be coupled directly or indirectly to the desired component of the assay according to methods well known in the art.

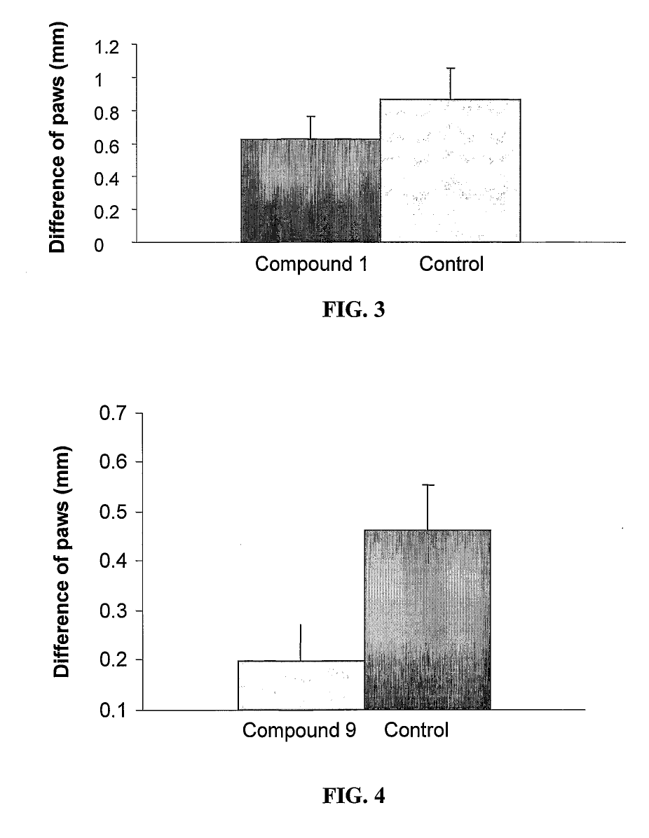

According to a further embodiment, the compounds of formula I inhibit leukocyte and neutrophil infiltration in vivo (see Example 10 herein below and FIG. 3).

The ability of compounds of the present invention to reduce leukocyte migration to sites of acute inflammation was evaluated in BALB/c mice using a thioglycolate-induced model of peritonitis. In this animal model, interactions of L- and P-selectin with HS-GAGs have been implicated in neutrophil infiltration (Nelson, R. M., 82:3253-3258, 1993; Xie, X. et al., J. Biol. Chem., 275:34818-34825, 2000).

Compounds according to formula I of the present invention were shown to efficiently inhibit leukocyte and neutrophil migration into the peritoneal cavity. The compounds were also shown to reduce lymphocyte migration, evaluated in mice using a model of Delayed Type Hypersensitivity (see Example 8 and FIG. 1; for the method see Lange-Asschenfeldt B. et al., Blood, 99:538-545, 2002).

Compounds of the present invention having the desired biological activity may be modified as necessary to provide desired properties such as improved pharmacological properties.

For diagnostic purposes, a wide variety of labels may be linked to the compounds, which may provide, directly or indirectly, a detectable signal. Thus, the compounds of the present invention may be modified in a variety of ways for a variety of end purposes while still retaining biological activity. In addition, various reactive sites may be introduced in the molecules for linking to particles, solid substrates, macromolecules, or the like.

Labeled compounds can be used in a variety of in vivo or in vitro applications. A wide variety of labels may be employed, such as radionuclides (e.g., gamma-emitting radioisotopes such as technetium-99 or indium-1111), fluorescent agents (e.g., fluorescein), enzymes, enzyme substrates, enzyme cofactors, enzyme inhibitors, chemiluminescent compounds, bioluminescent compounds, and the like. Those of ordinary skill in the art will know of other suitable labels for binding to the compounds, or will be able to ascertain such using routine experimentation. The binding of these labels is achieved using standard techniques common to those of ordinary skill in the art.

For in vivo diagnostic imaging to identify, for example, sites of inflammation, radioisotopes are typically used in accordance with well-known techniques.

The present invention relates also to pharmaceutical composition comprising compounds capable of inhibiting the interactions between glycosaminoglycans (GAGs), particularly heparan sulfate glycosaminoglycans (HS-GAGs), and GAG-binding viral proteins (GBVPs), particularly cytomegalovirus envelope glycoprotein B. A growing body of evidence points to the role of cell surface GAGs as the initial receptor in viral infection. Specifically, viruses such as Cytomegalovirus (CMV) (Boyle K A and Compton T 1998, J Virol. 72, 1826-1833) employ HS-GAGs for their initial step of infection. Attachment of human CMV at the cell surface is rapid and efficient in permissive as well as non-permissive cell types, suggesting that cellular receptors for CMV are widely distributed. Addition of exogenous heparin or the treatment of cells with heparinase blocks viral attachment and implicates the proteoglycan heparan sulfate in the initial interaction between virus and cell. For human CMV it was shown that the envelope glycoprotein B(gB) is an important mediator of virus entry that works, at least in part, via heparin sulfate binding (Boyle K A and Compton T 1998, J Virol. 72, 1826-1833).

The invention includes pharmaceutically acceptable salts of the compounds of the present invention. Pharmaceutically acceptable salts can be prepared by treatment with inorganic bases, for example, sodium hydroxide or inorganic/organic acids such as hydrochloric acid, citric acids and the like.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It is to be understood that, as used herein, references to the compounds according to formula I of the present invention are meant to also include the pharmaceutically acceptable salts thereof.

The pharmaceutical compositions of the present invention can be formulated for administration by a variety of routes including oral, rectal, transdermal, subcutaneous, intravenous, intramuscular, and intranasal. Such compositions are prepared in a manner well known in the pharmaceutical art and comprise as an active ingredient at least one compound according to formula I as described herein above, further comprising an excipient or a carrier.

During the preparation of the pharmaceutical compositions according to the present invention the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active ingredient to provide the appropriate particle size prior to combining with the other ingredients. If the active compound is substantially insoluble, it ordinarily is milled to a particle size of less than 200 mesh. If the active ingredient is substantially water soluble, the particle size is normally adjusted by milling to provide a substantially uniform distribution in the formulation, e.g.,about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methylcellulose. The formulations can additionally include lubricating agents such as talc, magnesium stearate, and mineral oil; wetting agents; emulsifying and suspending agents; preserving agents such as methyl- and propyl-hydroxybenzoates; sweetening agents; and flavoring agents.

The compositions of the invention can be formulated so as to provide quick, sustained or delayed release of the active ingredient after administration to the patient by employing procedures known in the art.

The compositions are preferably formulated in a unit dosage form, each dosage containing from about 0.1 to about 500 mg of a compound of formula I. The term “unit dosage forms” refers to physically discrete units suitable as unitary dosages for human subjects and other mammals, each unit containing a predetermined quantity of the active compound calculated to produce the desired therapeutic effect, in association with a suitable pharmaceutical excipient.

The active ingredient is effective over a wide dosage range and is generally administered in a therapeutically effective amount. It will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings; such materials include a number of polymeric acids and mixtures of polymeric acids with materials such as shellac, cetyl alcohol, and cellulose acetate.

The liquid forms in which the compositions of the present invention may be incorporated, for administration orally or by injection, include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insulation include solutions and suspensions in pharmaceutically acceptable aqueous or organic solvents, or mixtures thereof, and powders. The liquid or solid compositions may contain suitable pharmaceutically acceptable excipients as described above. Preferably the compositions are administered by the oral or nasal respiratory route for local or systemic effect. Compositions in preferably pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, preferably orally or nasally, from devices that deliver the formulation in an appropriate manner.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252 incorporated herein by reference in its entirety as if fully set forth herein. Such patches may be constructed for continuous, pulsatile, or on demand delivery of pharmaceutical agents.

Direct or indirect placement techniques may be used when it is desirable or necessary to introduce the pharmaceutical composition to the brain. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 incorporated herein by reference in its entirety as if fully set forth herein. Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions, which can transiently open the blood-brain barrier.

The compounds of general formula I inhibit cell-matrix and cell-cell interaction, thus inhibiting a cascade of events that lead to the development of certain diseases and disorders.

Thus, according to some aspects, the present invention provides a method for the treatment or prevention of diseases and disorders related to cell adhesion and cell migration mediated by HS-GAG-L-selectin interactions, comprising the step of administering to a subject in need thereof a therapeutically effective amount of a compound of the general formula I.

According to one embodiment, the pharmaceutical compositions according to the present invention are used for the treatment of diseases or disorders related to GAG-L-selectin interactions wherein the GAGs are selected from the group consisting of heparan sulfate (HS-GAG), heparin, chondroitin sulfate, dermatan sulfate, keratan sulfate, and derivatives and fragments thereof.

According to one currently preferred embodiment, the pharmaceutical compositions according to the present invention are used for the treatment of diseases or disorders related to HS-GAGs.

Anti-cell adhesion and anti-cell migration therapy has proven to be highly effective in the treatment of a number of diseases, disorders and conditions including inflammatory processes, autoimmune processes, cancer and tumor metastasis, and platelet-mediated pathologies.

A number of inflammatory disorders associated with L-selectin or involving selectin-mediated leukocyte flow along the blood stream may be treated with the pharmaceutical compositions of the invention. Treatable disorders include, but are not limited to, organ or tissue transplantation rejection (e.g., allograft rejection or autologous bone marrow transplantation), atherosclerosis, retinitis, cancer metastases, rheumatoid arthritis, acute leukocyte-mediated lung injury (e.g., adult respiratory distress syndrome), asthma, allergic rhinitis, allergic conjunctivitis, inflammatory lung diseases, restenosis, nephritis, acute and chronic inflammation, atopic dermatitis, psoriasis, contact dermal hypersensitivity, myocardial ischemia, and inflammatory bowel disease.

In preferred embodiments, the pharmaceutical compositions are used to treat inflammatory disorders associated with neutrophil infiltration, such as ischemia-reperfusion injury, acute pancreatitis, septic shock, uveitis, rheumatoid arthritis, and inflammatory bowel disease.

Reperfusion injury is a major problem in clinical cardiology. Therapeutic agents that reduce leukocyte adherence in ischemic myocardium can significantly enhance the therapeutic efficacy of thrombolytic agents. Thrombolytic therapy with agents such as tissue plasminogen activator or streptokinase can relieve coronary artery obstruction in many patients with severe myocardial ischemia prior to irreversible myocardial cell death. However, many such patients still suffer myocardial necrosis despite restoration of blood flow. This “reperfusion injury” is known to be associated with adherence of leukocytes to vascular endothelium in the ischemic zone.

Inflammatory bowel disease is a collective term for two similar diseases referred to as Crohn's disease and ulcerative colitis. Crohn's disease is an idiopathic, chronic ulceroconstrictive inflammatory disease characterized by sharply delimited and typically transmural involvement of all layers of the bowel wall by a granulomatous inflammatory reaction. Any segment of the gastrointestinal tract, from the mouth to the anus, may be involved, although the disease most commonly affects the terminal ileum and/or colon. Ulcerative colitis is an inflammatory response limited largely to the colonic mucosa and submucosa. Lymphocytes and macrophages are numerous in lesions of inflammatory bowel disease and may contribute to inflammatory injury.

Asthma is a disease characterized by increased responsiveness of the tracheobronchial tree to various stimuli potentiating paroxysmal constriction of the bronchial airways. The stimuli cause release of various mediators of inflammation that recruit basophils, eosinophils and neutrophils, which cause inflammatory injury.

Rheumatoid arthritis is a chronic, relapsing inflammatory disease that primarily causes impairment and destruction of joints. Rheumatoid arthritis usually first affects the small joints of the hands and feet but then may involve the wrists, elbows, ankles and knees. The arthritis results from interaction of synovial cells with leukocytes that infiltrate from the circulation into the synovial lining of the joints.

Atherosclerosis is a disease of arteries. The basic lesion, the atheroma, consists of a raised focal plaque within the intima, having a core of lipid and a covering fibrous cap. Atheromas compromise arterial blood flow and weaken affected arteries. Myocardial and cerebral infarcts are a major consequence of this disease. Macrophages and leukocytes are recruited to atheromas and contribute to inflammatory injury.

The pharmaceutical compositions of the present invention can be further used in the treatment of organ or graft rejection. Over recent years there has been a considerable improvement in the efficiency of surgical techniques for transplanting tissues and organs such as skin, kidney, liver, heart, lung, pancreas and bone marrow. Perhaps the principal outstanding problem is the lack of satisfactory agents for inducing immnunotolerance in the recipient to the transplanted allograft or organ. When allogeneic cells or organs are transplanted into a host, the host immune system is likely to mount an immune response to foreign antigens in the transplant (host-versus-graft disease) leading to destruction of the transplanted tissue. CD8+ cells, CD4 cells and monocytes are all involved in the rejection of transplant tissues.

A related use of the pharmaceutical compositions according to the present invention is in modulating the immune response involved in “graft versus host” disease (GVHD). GVHD is a potentially fatal disease that occurs when immunologically competent cells are transferred to an allogeneic recipient. In this situation, the donor's immunocompetent cells may attack tissues in the recipient.

Further use of the pharmaceutical compositions according to the present invention is for the treatment of cancer, both primary tumors and metastasis. For certain cancers to spread throughout a patient's body, a process of cell-cell adhesion, or metastasis, must take place. Specifically, cancer cells must migrate from their site of origin, the primary tumor, and gain access to a blood vessel to facilitate colonization at distant sites. A critical aspect of this process is adhesion of cancer cells (to platelets and to endothelial cells that line the blood vessel wall), a step prior to migrating into surrounding tissue. This process can be interrupted by the administration of compounds of the invention, which generally aid in blocking cell-cell adhesion.

Also embodied is the use of the pharmaceutical compositions according to the present invention in the treatment of leukemia, such as acute myeloid leukemia (AML), which involves extravasation of leukemic cells and tumor formation.

Also embodied in the present invention are methods useful for the treatment and prevention of diseases and disorders associated with angiogenesis. The term “angiogenesis” as used herein includes conditions involving abnormal neovascularization, such as tumor angiogenesis, and ophthalmologic disorders such as neovascular glaucoma ,diabetic retinopathy and macular degeneration, particularly age-related macular degeneration, reperfusion of gastric ulcer, and also for contraception or for inducing abortion at early stages of pregnancy.

A further use of the pharmaceutical compositions according to the present invention is in treating multiple sclerosis. Multiple sclerosis is a progressive neurological autoimmune disease that is thought to be the result of a specific autoimmune reaction in which certain leukocytes initiate the destruction of myelin, the insulating sheath covering nerve fibers.

Compounds of the general formula I were shown to inhibit the interaction between HS-GAG and envelope glycoprotein B of the cytomegalovirus, as described in Example 7 and Table 2. Accordingly, in some aspects, the present invention provides a method for the treatment or prevention of diseases and disorders related to cytomegalovirus attachment and entry, comprising the step of administering to a subject in need thereof a pharmaceutical composition comprising a therapeutically effective amount of at least one molecule having the general formula I.

It has also been found that compounds according to formula I of the present invention directly bind to HS-GAGs and may therefore be useful for treatment of disease conditions mediated by HS-GAGs. HS-GAG mediated conditions include those mediated by cell-cell, cell-virus, cell-matrix and cell-protein interactions. Examples of HS-GAG mediated conditions include virus attachment to cell, cell adhesion, platelet aggregation, lymphocyte adhesion and migration, and amyloid fibril formation.

According to another embodiment, the pharmaceutical compositions according to the present invention are useful for treatment or prevention of viral disorders such as hepatitis C and B, cytomegalovirus infection, respiratory syncytial virus infection, and AIDS.

According to yet another embodiment, the pharmaceutical compositions of the present invention are useful for the treatment or prevention of atherosclerosis, amyloid disorders including Alzheimer's disease and type II diabetes (non-insulin dependent diabetes mellitus), inflammatory and immune disorders, cancer, bone degradation, osteoporosis, osteoarthritis, tumor metastasis, and kidney disease including glomerulonephritis.

According to yet another embodiment, the pharmaceutical compositions of the present invention are used for the treatment or prevention of coagulation disorders. The compounds of the present invention may be useful for counteracting the actions of heparin and other anticoagulant glycosaminoglycans on thrombin and Factor Xa activity, and may affect other coagulation proteins as well. Heparin is used routinely for anticoagulation. It is often necessary to reverse the effects of heparin when anticoagulation has reached a stage at which hemorrhage becomes a threat, notably after the routine use of heparin for anticoagulation during cardiopulmonary bypass, and in patients who develop an endogenous heparin-like coagulation inhibitor. Currently, the only FDA-approved heparin antidote available is Protamine. Protamine is a mixture of basic proteins from fish sperm nuclei that contains a high concentration of the amino acid arginine. When injected into a person who has been treated with heparin, Protamine complexes rapidly to the heparin, thereby neutralizing its activity. Although Protamine is effective in humans against unfractionated heparin, it is not effective against low molecular weight heparins or against the non-heparin glycosaminoglycan anticoagulant Orgaran®), i.e., a mixture of chondroitin sulfate/heparan sulfate/derinatan sulfate. Protamine also has numerous side effects including pulmonary hypotension, that are difficult to control and constitute significant health risks to the patient. Also, since Protamine is obtained from a natural source, it is a poorly-defined and potentially variable product, and therefore dosage determination can be problematic. The compounds of the invention can be useful in neutralization of unfractionated heparin, low molecular weight heparin, or Orgaran®.

Additional possible use for the compounds of the present invention is to block the uptake and clearance of heparin by blocking heparin receptors in tissues without binding to circulating heparin, and thus to prolong the half-life of heparin in the circulation. Use of the compounds of the invention would reduce the frequency of administration of heparin, as well as the amount needed. This could be especially useful for home-based therapy with low molecular weight heparin, which is administered by subcutaneous injection and is becoming the standard post-hospitalization anticoagulation treatment.

It is to be understood that while the compounds according to formula I of the present invention were selected for their capacity to inhibit binding of L-selectin to HS-GAGs, and that this property contributes to their medical activity, it cannot, however, be excluded that the compounds are also exerting their favorable medical effects, either in parallel or in tandem, through additional mechanisms of action. Thus, the skilled practitioner of this art will appreciate that one aspect of the present invention is the description of novel pharmaceutical compositions, and that Applicants intend not to be bound by a particular mechanism of action that may account for their prophylactic or therapeutic effects.

The principles of the invention, providing compounds described here for the first time with regard to their pharmaceutical uses and some novel compounds, all these compounds being capable of inhibiting GAG-L-selectin interactions, their pharmaceutical compositions and uses thereof according to the present invention, may be better understood with reference to the following non-limiting examples.

### EXAMPLES

The structural formulas of the inhibitor compounds herein identified as Compounds Nos. 1-9 and of the novel compounds herein identified as Compounds Nos. 2010, 2011 and 2012 are presented in the Appendix just before the claims.

### Example 1.

### General Procedure for Synthesis of Compounds of Formula I

The quinazolinecarboxamide derivatives are prepared by amidation of the corresponding quinazolinecarboxylic acid according to the following general scheme of the reaction:

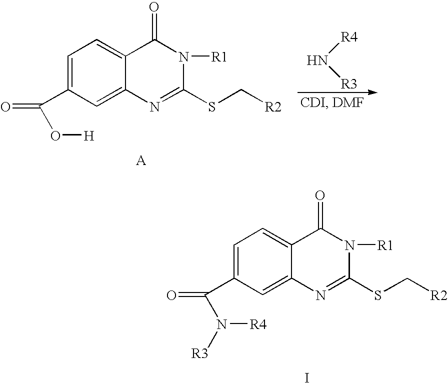

The starting acids of the formula A have been synthesized as described (Ivatchtchenko, A. V., Kovalenko, S. M. and Drushlyak, O. G., J. Comb. Chem. 5, 775-788, 2003). To the solution of the acid A (2 mmol) in DMF (5 ml), 2.4 mmole of carbonyl diimidazole (CDI) was added drop wise and the mixture was stirred for one hour at 50° C. Then, 2.8 mmol of amine was added to the reaction mixture and the resulting solution was treated in ultrasonic bath with heating at 50° C. and stirring for 3 hours. After the reaction mixture returned to room temperature, some water was added. The resulted oil was solidified with isopropyl alcohol and washed with acetonitrile. The yields of the compounds of formula I are in the range of 70-90%.

### Example 2.

### Synthesis of 2-[[(6-nitro-4H-1,3-benzodioxin-8-yl)methyl]thio]-3-(2-propenyl)-3,4-dihydro-4-oxo-N-[3-(4-morpholinyl)propyl]-7-quinazoline-carboxamide (Compound No. 2010)

The synthesis of the title compound was performed as follows: To a solution of 2-[[[6-nitro-4H-1,3-benzodioxin-8-yl)methyl]thio]-3-(2-propenyl)-3,4-dihydro-4-oxo-7-quinazolinecarboxylic acid 0.9 g (2 mmol) in DMF (5 ml), 2,4 mmol of CDI was added drop wise and the mixture was stirred for one hour at 50° C. Then 2.8 mmol of 4-(3-aminopropyl)morpholine 0.40 g (0.41 ml) was added to the reaction mixture and the resulting solution was treated in ultrasonic bath with heating at 50° C. and stirring for 3 hours. After the reaction mixture returned to room temperature, some water was added. The resulting oil was solidified with isopropyl alcohol and washed with acetonitrile. The title compound was obtained in 90% yield. NMR spectra was as follows. ¹H NMR (DMSO-d₆) δ (ppm): 2.30 (s, 6H), 2.80 (d, 2H), 3.55(s, 4H), 4.50 (s, 2H), 4.52 (s, 2H), 4.90 (s, 2H), 5.15 (dd, 2H), 5.45 (s, 2H), 5.90 (m, 1H), 7.84 (d, J=7.6 Hz, 1H), 8.02 (s, 1H), 8.10 (s, 1H), 8.13 (d, J=7.6 Hz, 1H), 8.40 (s, 1H), 8.78 (s, 1H). Mass spectra (TOF [time-of-flight]): m/z 582 (M+H)⁺.

### Example 3.

### Synthesis of 2-[[(5-acetyl-2-methoxyphenyl)methyl)thio]-3-(phenylmethyl) -3,4-dihydro-4-oxo-N-[3-(1H-imidazol-1-yl)propyl]-7-quinazolinecarboxamide (Compound No. 2011).

The synthesis of the title compound was performed as follows: To a solution of 2-[[(5-acetyl-2-methoxyphenyl)methyl]thio]-3-(phenylmethyl)-3,4-dihydro-4-oxo -7-quinazolinecarboxylic acid 0.95 g (2 mmol) in DMF (5 ml), 2.4 mmol of CDI was added drop wise and the mixture was stirred for one hour at 50° C. Then 2.8 mmol of 1-(3-aminopropyl)imidazole 0.35 g (0.33 ml) was added to the reaction mixture and the resulting solution was treated in ultrasonic bath with heating at 50° C. and stirring for 3 hours. After the reaction mixture returned to room temperature some water was added. The resulting oil was solidified with isopropyl alcohol and washed with acetonitrile. The ttitle compound was obtained in 75% yield. NMR spectra was as follows. MS (TOF): m/z 583 (M+H)⁺

¹H NMR (DMSO-d₆) δ (ppm): 2.00 (m, 2H), 2.45 (s, 3H), 3.35 (m, 2H), 3.85 (s, 3H), 4.05 (m, 2H), 4.55 (s, 2H), 5.30 (s, 2H), 6.90 (s, 1H), 7.15 (d, 1H), 7.30 (m, 6H), 7.65 (s, 1H), 7.90 (m, 2H), 8.20 (m, 3H), 8.75 (s, 1H).

### Example 4.

### Synthesis of 2-[[(5-acetyl-2-methoxyphenyl)methyl]thio]-3-(phenylmethyl) -3,4-dihydro-4-oxo-N-(1-ethyl-piperidin-4-yl)-7-quinazoline-carboxamide (Compound No. 2012).

The synthesis of the title compound was performed as follows: To a solution of 2-[[(5-acetyl-2-methoxyphenyl)methyl]thio]-3-(phenylmethyl)-3,4-dihydro-4-oxo -7-quinazolinecarboxylic acid 0.95 g (2 mmol) in DMF (5 ml), 2,4 mmol of CDI was added drop wise and the mixture was stirred for one hour at 50° C. Then 2.8 mmol of 1-ethyl-4-amino piperidine was added to the reaction mixture and the resulting solution was treated in ultrasonic bath with heating at 50° C. and stirring for 3 hours. After the reaction mixture returned to room temperature, some water was added. The resulting oil was solidified with isopropyl alcohol and washed with acetonitrile. The title compound was obtained in 85% yield. MS (TOF): m/z 586 (M+H)⁺

¹H NMR (DMSO-d₆) δ (ppm): 1.00 (t, 3H), 1.60 (m, 2H), 1.80 (m, 2H), 1.95 (m, 2H), 2.35 (m, 2H), 2.45 (s, 3H), 3.85 (m, 1H), 3.90 (s, 3H), 4.55 (s, 2H), 5.30 (s, 2H), 7.10 (d, 1H), 7.25 (m, 5H), 7.85 (t, 2H), 8.15 (m, 3H), 8.40 (s, 1H).

### Example 5.

### Pharmaceutical Compositions

The pharmaceutical compositions of the present invention are illustrated by the following formulation examples:

(i) Formulation 1

Hard gelatin capsules containing the following ingredients are prepared:

| Ingredient | Quantity (mg/capsule) |
|---|---|
| Active Ingredient | 30.0 |
| Starch | 305.0 |
| Magnesium stearate | 5.0 |

The above ingredients are mixed and filled into hard gelatin capsules in 340 mg quantities.

(ii) Formulation 2

A tablet formula is prepared using the ingredients below:

| Ingredient | Quantity (mg/tablet) |
|---|---|
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

(iii) Formulation 3

A dry powder inhaler formulation is prepared containing the following components:

| Ingredient | Weight % |
|---|---|
| Active Ingredient | 5.0 |
| Lactose | 95.0 |

The active ingredient is mixed with the lactose and the mixture is added to a dry powder inhaling-appliance.

(iv) Formulation 4

Tablets, each containing 30 mg of active ingredient, are prepared as follows:

| Ingredient | Quantity (mg/tablet) |
|---|---|
| Active Ingredient | 30.0 |
| Starch | 45.0 |
| Microcrystalline cellulose | 35.0 |
| Polyvinylpyrrolidone | 4.0 |
| (as 10% solution in water) | |
| Sodium carboxymethyl starch | 4.50 |
| Magnesium stearate | 0.5 |
| Talc | 1.0 |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° C. to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

(v) Formulation 5

Capsules, each containing 40 mg of the active ingredient, are made as follows:

| Ingredient | Quantity (mg/capsule) |
|---|---|
| Active Ingredient | 40.0 |
| Starch | 109.0 |
| Magnesium stearate | 1.0 |

The active ingredient, starch, and magnesium stearate are blended, passed through a No. 20 mesh U.S. sieve, and filled into hard gelatin capsules in 150 mg quantities.

(vi) Formulation 6

Suppositories, each containing 25 mg of active ingredient, are made as follows:

| Ingredient | Quantity (mg) |
|---|---|
| Active Ingredient | 25.0 |
| Saturated fatty acid glycerides | 2000.0 |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

(vii) Formulation 7

Suspensions, each containing 50 mg of an active ingredient per 5.0 ml dose, are made as follows:

| Ingredient | Quantity (mg) | |
|---|---|---|
| Active Ingredient | 50.0 | mg |
| Xanthan gum | 4.0 | mg |
| Sodium carboxymethyl cellulose (11%) | 50.0 | mg |
| Microcrystalline cellulose (89%) | | |
| Sucrose | 1.75 | g |
| Sodium benzoate | 10.0 | mg |
| Flavor and Color | q.v. | mg |
| Purified water | to 5.0 | ml |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

(viii) Formulation 8

Capsules, each containing 15 mg of an active ingredient, are made as follows:

| Ingredient | Quantity (mg/capsule) |
|---|---|
| Active Ingredient | 15.0 |
| Starch | 407.0 |
| Magnesium stearate | 3.0 |

The active ingredient, cellulose, starch, and magnesium stearate are blended, passed through a No. 20 mesh U.S. sieve, and filled into hard gelatin capsules in 425 mg quantities.

(ix) Formulation 9

An intravenous formulation is prepared as follows:

| Ingredient | Quantity | |
|---|---|---|
| Active Ingredient | 250.0 | mg |
| Isotonic saline | 1000 | ml |

(x) Formulation 10

A topical formulation is prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solidified.

### Example 6.

### In Vitro Assay for Determining Inhibition of L-selectin Binding to HS-GAGs by Inhibitor Compounds of the Formula I

An in vitro assay was used to assess the ability of test compounds according to formula I to inhibit the interactions of L-selectin with HS-GAGs. The assay was suitable for determining the concentration required for 50% inhibition (IC-50) for each specific compound. In the assay, the GAG used was heparin. Thus, porcine intestinal mucosa heparin conjugated to Bovine Serum Albumin (Heparin-BSA; Sigma Cat. No. H0403) at 5 mg/ml in Phosphate-Buffered Saline (PBS; pH 6.5) was added to a 96-well polystyrene ELISA plate (NUNC Cat. No. 442404; 0.1 ml per well) and incubated over night at 4° C. Following the incubation, the plate was washed thoroughly, by immersion, with de-ionized water and PBS (pH 6.5). The ELISA plate was then blocked with BSA (ICN Cat. No.160069, 3%, 200 μl per well) for 1 hour at room temperature. Following blocking, the plate was washed with de-ionized water, and then with PBS (pH 6.5) containing Tween 20 (Sigma Cat. No. P-1379, 0.05%). Compounds were synthesized or purchased from suppliers of chemical compounds such as ChemDiv Labs (San Diego, Calif.), dissolved in DMSO, diluted in PBS and added to the wells at various concentrations in the range of 0.01 to 300 μM. Recombinant Human L-Selectin/IgG (Research and Development Systems Cat. No.728-LS) dissolved in PBS supplemented with BSA (0.1%) and calcium chloride (1 mM) was added to the ELISA plate (100 μl per well) and incubated for 60 minutes at room temperature with shaking. Following incubation, the plate was washed with de-ionized water and three times with PBS (pH 6.5) containing Tween 20. Anti-Human IgG Peroxidase Conjugate (1:5000; Sigma Product No. A8667) diluted in PBS supplemented with BSA (0.1%) and calcium chloride (1 mM) was added to the ELISA plate (100 μl per well) and incubated for 30 minutes at room temperature with shaking. The plate was then washed with de-ionized water and three times with PBS (pH 6.5) containing Tween 20. The peroxidase substrate chromogen tetramethyl benzidine (TMB; Dako Cat. No. S1599) was added (100 μl per well) to the ELISA plate and incubated at room temperature. After 15 minutes, ELISA Stop Solution (hydrochloric acid 1N, sulfuric acid 3N) was added (200 μl per well) to stop the peroxidase catalyzed colorimetric reaction. The Optical Density (OD) of the samples was measured at 450 nm using an ELISA plate reader (Dynatech MR5000). Data were analyzed with Graphpad Prism software and IC-50 values were established.

It was established that compounds of Formula I had inhibitory activity in the above assay. Examples of Inhibitor Compounds are given in Table 1.

TABLE 1
Inhibition of L-Selectin Binding to Heparin by
Selected Quinazolinecarboxamide Derivatives

| | | % | % |
|---|---|---|---|
| Compound | | Inhibition at 100 | Inhibition at 30 |
| Number | IC-50 | microMolar conc. | microMolar conc. |
| 1 | 7 ± 1 | | |
| 2 | 24 ± 1.2 | | |
| 3 | 22 ± 1.2 | | |
| 4 | 14 ± 1 | | |
| 5 | | 52 | 50 |
| 6 | | | 30 |
| 7 | | | 83 |
| 8 | | | 59 |
| 9 | 21 ± 1.6 | | |
| 10 | 22 ± 1.5 | | |

All assays were repeated at least twice and representative results are shown.

### Example 7.

### Inhibition of CMV Envelope Glycoprotein B Binding to Immobilized Heparin by Inhibitor Compounds of Formula I

Porcine intestinal mucosa heparin conjugated to Bovine Serum Albumin (Heparin-BSA; Sigma Cat. No. H0403) at 5 mg/ml in Phosphate-Buffered Saline (PBS; pH 6.5) was added to a 96-well polystyrene ELISA plate (NUNC Cat. No. 442404; 0.1 ml per well) and incubated over night at 4° C. Following the incubation, the plate was washed consecutively, by immersion, with de-ionized water and PBS (pH 6.5). The ELISA plate was then blocked with BSA (ICN Cat. No. 160069, 3%, 200 μl per well) for 1 hour at room temperature. Following blocking, the plate was washed with de-ionized water, and then with PBS (pH 6.5) plus Tween 20 (0.05%). CMV envelope glycoprotein B (Research Diagnostics, INC. Cat. No. RDI-RCMVAG-B) dissolved in PBS (supplemented with BSA (0.1%)) was added to the ELISA plate (100 μl per well) and incubated for 60 minutes at room temperature with shaking. Following incubation, the plate was washed with de-ionized water and with PBS (pH 6.5) plus Tween. Mouse anti-human Cytomegalovirus gB antibody (Research Diagnostics, INC. Cat. No. RDI-CMVG Babm) diluted in PBS (supplemented with BSA(0.1%)), 1:2000, was added to the ELISA plate (100 μl per well) and incubated for 90 minutes at room temperature with shaking. Following the incubation, the plate was washed with de-ionized water and PBS (pH 6.5) plus Tween. Goat anti-Mouse IgG (H&L) Peroxidase Conjugated antibody (Chemicon International, Inc. Cat. No. AP124P) diluted in PBS (supplemented with BSA (0.1%)), 1:1000, was added to the ELISA plate (100 μl per well) and incubated for 30 minutes at room temperature with shaking. Following the incubation, the plate was washed with de-ionized water and with PBS (pH 6.5) plus Tween. The peroxidase substrate chromogen, TMB (Dako Cat. No. S1599) was added (100 μl per well) to the ELISA plate and incubated at room temperature. After 15 minutes, ELISA Stop Solution (hydrochloric acid iN, sulfuric acid 3N) was added (200 μl per well) to stop the peroxidase catalyzed colorimetric reaction. The Optical Density (OD) of the samples was measured at 450 nm using an ELISA plate reader (Dynatech MR5000).

The CMV envelope glycoprotein B (GBVP) binding assay described above was used to screen a synthetic chemical compound collection on 96-well plates. The compound collection was purchased from ChemDiv Inc. (San Diego, Calif.). Compounds were dissolved in DMSO at 10 mM final concentration and further diluted prior to assay. DMSO concentration in the screening well was up to 2%. Individual compounds at a final concentration of 30 or 100 μM were co-incubated with the GBVP on plates containing immobilized heparin and, following washing, bound GBVP was detected with anti-CMV GBVP antibody and secondary antibody conjugated to horseradish peroxidase, as described above. Following color development, the % inhibition compared to control (no compound) for every compound was determined. Compounds that inhibited at least 30% of the signal were scored as hits. The Inhibitor Compounds are listed in Table 2.

TABLE 2
Inhibition of CMV envelope glycoprotein B binding
to immobilized heparin by Compounds 1-4

| Compound | % Inhibition at 100 | % Inhibition at 30 |
|---|---|---|
| Number | microMolar conc. | microMolar conc. |
| 1 | | 33 |
| 2 | 86 | |
| 3 | 88 | 85 |
| 4 | | 79 |

### Example 8.

### Delayed-type Hypersensitivity (DTH)

BALB/c mice (Velaz, Prague, Czech Republic; 8 weeks old; 15 animals per group) were sensitized by topical application of a 2% oxazolone (4-ethoxymethylene -2-phenyl-2-oxazoline-5-one; Sigma, St Louis, Mo.) solution in acetone/olive oil (4:1 vol/vol) to shaved abdomen (50 μl) and to each paw (5 μl) (Lange-Asschenfeldt B. et al., Blood 99:538-545, 2002). Five days after sensitization, right ears were challenged by topical application of 10 μl of a 1% oxazolone solution, whereas left ears were treated with vehicle alone. Compounds were administered 1 hr prior to challenge. The extent of inflammation was measured 24 hours after challenge, using the mouse ear-swelling test. Animals were numbered (tail marking) and weighed and the thickness of both ears was recorded with a constant-loading dial micrometer (Mitutoyo, Tokyo). The unpaired Student t-test was used for statistical analyses.

As shown in FIG. 1, Compound No. 2 (at a dose of 25 mg/kg, administered per os) inhibited DTH 24 hours after challenge. Data were statistically significant as determined by Student's t test with p>0.05.

### Example 9.

### Carrageenan-induced Paw Edema

Acute edema was induced in the left hind paw of BALB/c mice (12 mice/group) by injecting 0.02 ml of freshly prepared solution of 2% carrageenan (Sigma) after 60 min of test compound administration (Torres, S. R. et al., European Journal of Pharmacology 408: 199-211, 2000). The right paw received 0.02 ml of saline, which served as a control. Carrageenan was injected under the plantar region of right hind paw and the paw thickness was measured at 2,4 and 24 hours after carrageenan challenge using a Mitutoyo engineer's micrometer expressed as the difference between right and left pad as mean ±SEM. As shown in FIG. 3, oral administration of 1 mg/kg of Compound No. 1 inhibited paw edema and the data were statistically significant as determined by Student's t test with p>0.001. As shown in FIG. 4, oral administration of 50 mg/kg of Compound No. 9 inhibited paw edema and the data were statistically significant as determined by Student's t test with p>0.001.

### Example 10.

### A Model of Leukocyte and Neutrophil Infiltration into Mouse Peritoneum

BALB/c mice (Velaz, Prague; 6 weeks old, ˜20 g in weight, 15 mice/group) received intraperitoneal injection of an inhibitor compound in 0.2 ml DMSO/Tween/sterile saline 1 hour before administration of thioglycollate (Sigma). Control groups received vehicle and sham controls received no thioglycollate. Mice were injected intraperitoneally with 1 ml of 3% thioglycollate broth (Xie, X. et al., J. Biol. Chem., 275, 34818-34825, 2000). Mice were sacrificed after 3 hours, and the peritoneal cavities were lavaged with 5 ml of ice-cold saline containing 2 mM EDTA to prevent clotting. After red blood cell lysis, leukocytes were counted in a hemocytometer. Neutrophils were counted after staining with Türck's reagent (Merck, Darmstadt, Germany). Data was expressed as mean ±SEM, and statistical analysis was performed by Student t test. A value of p<0.05 was taken to denote statistical significance.

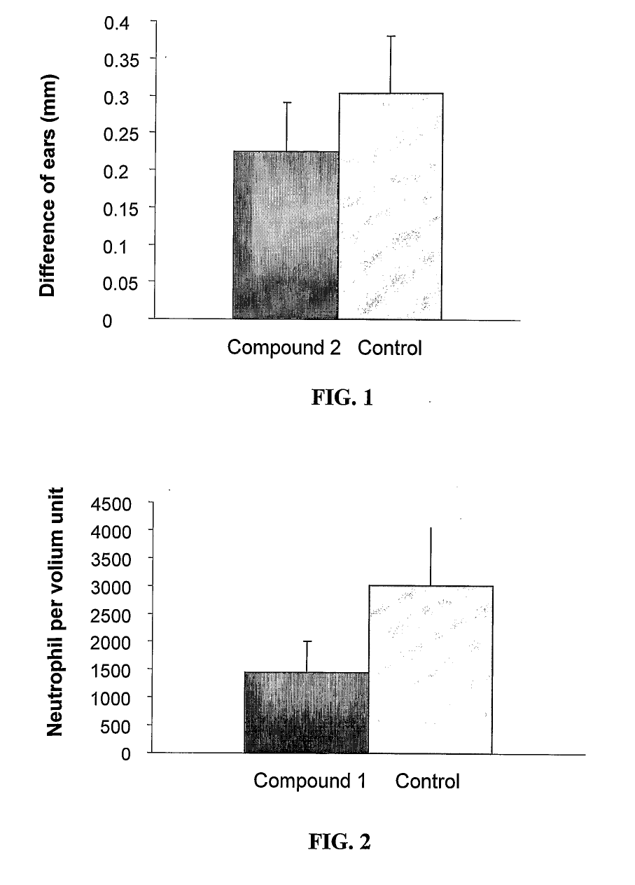

Thioglycollate administration induced approximately 3-fold increase in leukocyte accumulation in the peritoneal cavity. Leukocyte migration into the peritoneal cavity was inhibited efficiently by administration of Compound No. 1 as shown in FIG. 2. Similar results were obtained when the neutrophil counts were determined. Compound No. 1 at 10 mg/kg i.p. inhibited neutrophil accumulation to 48% of control value and the data were statistically significant as determined by Student's t test with p>0.001. It is well known that leukocyte migration and infiltration in vivo is a hallmark of inflammatory, autoimmune and other disorders. The ability of the inhibitor compounds of the invention to inhibit leukocyte infiltration in vivo indicates, therefore, the potential therapeutic applications of these compounds for treatment of these disorders.

### Example 11.

### Trinitrobenzene Sulfonic Acid (TNBS)-induced Colitis Model

Control BALB/c mice (male; aged 6-8 weeks at start of experiment; 12 per group; Harlan, Israel) are injected intraperitoneally (IP) with Test Compound (TC) Vehicle (Tween 80, 5%, 200 μl). Experimental mice (12 per group) are injected IP with TC (10 mg/kg, or 35 mg/kg in 200 μl). The control and experimental mice are injected once per day for 7 successive days. 24 hours after the first IP injection, colitis is induced in the control, experimental, and in an untreated group by intra-rectal administration of TNBS (150 mg/kg dissolved in NaCl (0.9%): EtOH (50%) (1:1; 80 μl mouse). All of the mice are killed by cervical dislocation 7 days after TNBS administration. The colons of the mice are examined under a dissecting microscope (X5) to evaluate the macroscopic lesions on a scale of 0 to 10 (colonic damage score). Gross colonic damage is graded according to Reuter et al. (Reuter B K, Asfaha S, Buret A, Sharkey K A, Wallace J L. Exacerbation of inflammation-associated colonic injury in rat through inhibition of cyclooxygenase-2. J Clin. Invest. 98:2076-85 (1996)), using the combined values of the four standard macroscopic parameters: degree of colonic ulcerations (scale from 0—completely normal, to 10—most severe); intestinal and peritoneal adhesions (0 to 2); diarrhea (0 to 1); and thickness (0 to 1). The total score is the arithmetic sum of the four scores. The evaluation is performed in a blind procedure.

### Example 12.

### Experimental Autoimmune Encephalomyelitis (EAE)

In autoimmune conditions, T cells reactive to self-antigens escape elimination in the thymus and are activated in the periphery, where they can provoke damage in specific organs. EAE, a model of autoimmune disease that is induced in Lewis rats, bears many similarities to the human disease Multiple Sclerosis (MS). T cells found in brain lesions of MS patients have T cell receptor (TCR) junctional rearrangements that are identical to T cells found in the spinal cords of Lewis rats immunized with a peptide of myelin basic protein (MBP) p87-99. In addition, a major T and B cell response in MS patients is directed to MBP p87-99.

EAE is induced in rats by immunization with MBP p87-99 or in SJL/J mice by immunization with PLP 139-151 peptide. The Inhibitor Compounds are administered once daily by intraperitoneal injection for 3 consecutive days starting 1 day before the appearance of EAE symptoms (day 12 after EAE induction). Following the encephalitogenic challenge, mice are observed daily and clinical manifestations of EAE are scored on a scale of 0-5. The degree of clinical disease is scored as follows: 0=no signs; 1=loss of tail tonicity; 2=paralysis of hind limbs; 3=paralysis of all four limbs 4=quadriplegic animal in a moribund state; 5=death. Treatment with the Inhibitor Compounds is expected to result in significantly reduced clinical severity and incidence of EAE.

### Example 13.

### Methods to Measure the Counteracting Actions of Heparin and Other Anticoagulant GAGs on Coagulation

One or more of the following assays can be used:

(i) In Vitro Effect of the Test Compounds on Reversal of Factor Xa Activity.

Solutions of Lovenox (Rhone Poulenc Rohrer), Orgaran (Organan), or unfractionated heparin (Sigma) are prepared in 0.32% sodium citrate or in normal human plasma to contain 0.5 U/ml anti Factor Xa activity. Calibrations are made against the standards provided by the Stachrom Heparin (Diagnostica Stago) assay kit (Dignac M et al, Nouv. Res. Fr Hematol. 35:545-549, 1994). The heparin/ATIII complex is allowed to form at 37° C. for 2 minutes, inhibitor Compound is added, the mixture is incubated for an additional 1-5 minutes, then Factor Xa is added, and finally the chromogenic substrate is added for 1 minute, and the absorbance is read at 405 nm. The increase in absorbance of the heparinized control vs. that of the test sample is divided by the difference in the absorbance at 405 between the heparinized control and the control without heparin, to obtain the % reversal.

(ii) In Vitro Effect of the Test Compounds on Reversal of Inhibition of thrombin Activity by Unfractionated Heparin.

Plasma is obtained from normal donors. Thrombin concentration (human alpha thrombin, Enzyme Research Laboratories, South Bend, Ind.) is standardized to produce a clotting time of 20-22 seconds. Heparin is added at 0.5 IU anti-thrombin activity/ml. The clotting time for heparin alone is approximately 3 minutes. To test the effects of the compounds in this system, one minute after addition of heparin to the plasma, the compounds are added in concentrations ranging from 0.1-100 μM. After one minute, thrombin is added and the clotting time is determined.

(iii) In Vivo Effect of Test Compounds in Reversing Effects of Lovenox on Factor Xa Activity.

Rats (300-400 g) are anesthetized with ketamine/acepromazine and are cannulated in the left jugular vein and right femoral vein. Blood is drawn immediately before injection of the low molecular weight heparin Lovenox to establish baseline Factor Xa activity. Lovenox (43 IU anti-FXa activity/kg in 0.1 ml saline, based on suggested dosage for humans) is injected through the jugular catheter, followed immediately by 0.2 ml of saline. Blood (0.1 ml) is collected into sodium citrate from the femoral vein every 30 seconds for 3 min. The compound is injected at 3 min through the jugular catheter in 0.1 ml of phosphate-buffered saline, followed by a 0.2 ml saline flush. Compounds are administered and blood collection is immediately resumed every 30 seconds until 10 minutes after the initial Lovenox injection, then at 15, 20, 25 and 30 min. The samples are centrifuged to obtain plasma and are assayed for residual Lovenox by assay of anti-Factor Xa activity by the Stachrom Heparin test kit. Absorbance at 405 nm is measured after a 1-minute incubation with the chromogenic Factor Xa substrate.

It will be appreciated by persons skilled in the art that the present invention is not limited by what has been particularly shown and described herein above. Rather the scope of the invention is defined by the claims that follow.

### APPENDIX

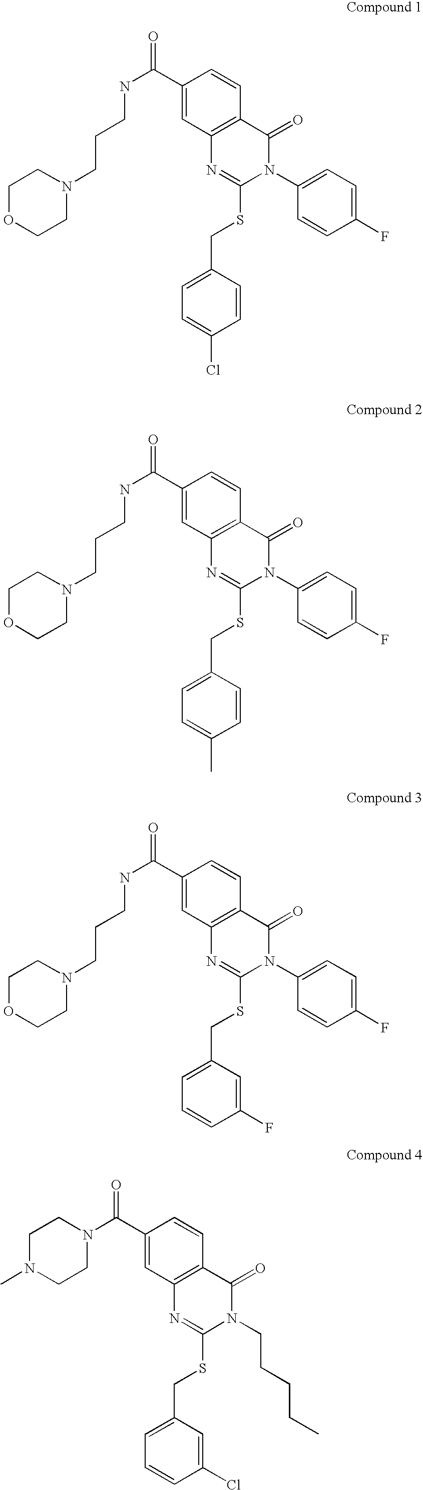

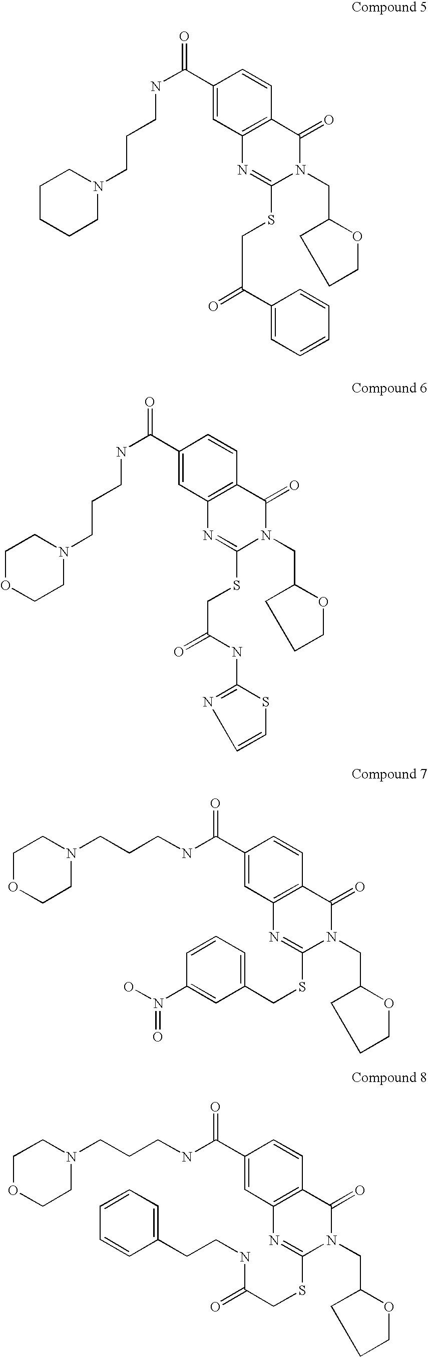

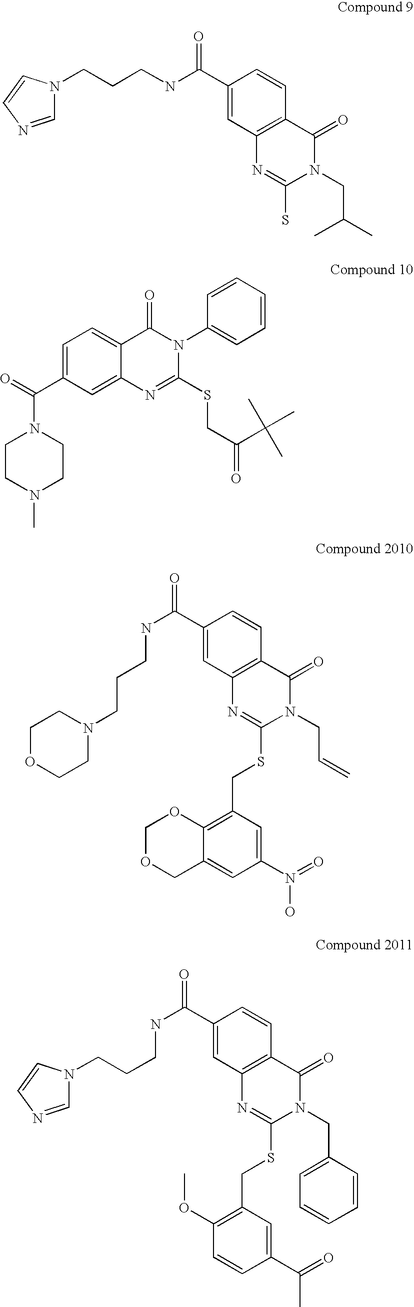

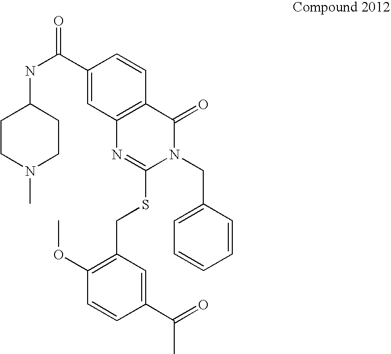

1. A pharmaceutical composition comprising a pharmaceutically acceptable diluent or carrier and an active ingredient of the general formula I: 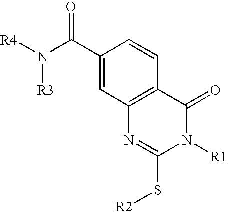
wherein:
R₁ is optionally substituted hydrocarbyl or heterocyclyl;
R₂ is selected from the group consisting of H, (C₁-C₁₂)alkyl, (C₆-C₁₄)aryl-CH₂-, heteroaryl-CH₂-, alkylcarbonyl-CH₂-, (C6-C₁₄) arylcarbonyl-CH₂- and heteroarylcarbonyl-CH₂-;
R₃ and R₄ each is selected from the group consisting of hydrogen, C₁-C₆ alkyl, (C₁-C₆)alkoxy(C₁-C₆)alkyl, and C₁-C₆ alkyl substituted by a group containing a basic nitrogen atom or by a 5-7 membered heterocyclic ring containing one or two heteroatoms, one of them being a basic nitrogen atom, or R₃ and R₄ together with the nitrogen atom to which they are attached form a 5-7 membered saturated heterocyclic ring containing one or two basic nitrogen atoms, optionally substituted on the additional nitrogen atom;
and pharmaceutically acceptable salts thereof.

2-17. (canceled)

18. The pharmaceutical composition according to claim 1, wherein the compound of formula I is selected from the group consisting of:
2-[[(4-chlorophenyl)methyl]thio]-3-(4-fluorophenyl)-3,4-dihydro -4-oxo-N-[3-(4-morpholinyl)propyl]-7-quinazolinecarboxamide [Compound No. 1];
2-[[(4-methylphenyl)methyl]thio]-3-(4-fluorophenyl)-3,4-dihydro -4-oxo-N-[3-(4-morpholinyl)propyl]-7-quinazolinecarboxamide [Compound No. 2];
2-[[(3-fluorophenyl)methyl]thio]-3-(4-fluorophenyl)-3,4-dihydro -4-oxo-N-[3-(4-morpholinyl)propyl]-7-quinazolinecarboxamide [Compound No. 3];
2-[(2-oxo-2-phenylethyl)thio]-3-(4-fluorophenyl)-3,4-dihydro-4-oxo-N-[3-(4-morpholinyl)propyl]-7-quinazolinecarboxamide [Compound No. 5]; and
2-[[2-[[(3-chlorophenyl)methyl]thio]-3-pentyl-3,4-dihydro-4-oxo -N-(4-methylpiperazinyl)-7-quinazolinecarboxamide [Compound No. 4].

19. The pharmaceutical composition according to claim 1 wherein the compound of formula I is 2-[[(6-nitro-4H-1,3-benzodioxin-8-yl)methyl]thio]-3-(2-propenyl) -3,4-dihydro-4-oxo-N-[3-(4-morpholinyl)propyl]-7-quinazolinecarboxamide (Compound No. 2010).

20. The pharmaceutical composition according to claim 1 wherein the compound of formula I is 2-[[(5-acetyl-2-methoxyphenyl)methyl)thio]-3-(phenylmethyl)-3,4-dihydro -4-oxo-N-[3-(1H-imidazol-1-yl)propyl]-7-quinazolinecarboxamide (Compound No. 2011).

21. The pharmaceutical composition according to claim 1, wherein the compound of formula I is 2-[[(5-acetyl-2-methoxyphenyl)methyl]thio]-3-(phenylmethyl)-3,4-dihydro -4-oxo-N-(1-ethyl-piperidin-4-yl)-7-quinazoline-carboxamide (Compound No. 2012).

22. The pharmaceutical composition according claim 1, for the treatment or prevention of inflammatory or autoimmune diseases, disorders or conditions related to cell adhesion or cell migration mediated by heparan sulfate glycosaminoglycans (HS-GAGs).

23-28. (canceled)

29. The pharmaceutical composition according to claim 1, for modulating the anticoagulant activity of glycosaminoglycans.

30. The pharmaceutical composition according to claim 29, wherein the glycosaminoglycan is heparin.

31. The pharmaceutical composition according to claim 1, capable of inhibiting the interaction of glycosaminoglycans with selectins.

32. The pharmaceutical composition according to claim 1, capable of inhibiting neutrophil infiltration in vivo.

33-42. (canceled)

43. The compound 2-[[(6-nitro-4H-1,3-benzodioxin -8-yl)methyl]thio]-3-(2-propenyl)-3,4-dihydro-4-oxo -N-[3-(4-morpholinyl)propyl]-7-quinazoline carboxamide (Compound No. 2010).

44. The compound 2-[[(5-acetyl-2-methoxyphenyl) methyl)thio]-3-(phenyl-methyl)-3,4-dihydro-4-oxo -N-[3-(1H-imidazol-1-yl)propyl]-7-quinazoline-carboxamide (Compound No. 2011).

45. The compound 2-[[(5-acetyl-2-methoxyphenyl) methyl]thio]-3-(phenyl-methyl)-3,4-dihydro-4-oxo -N-(1-ethyl-piperidin-4-yl)-7-quinazolinecarboxamide (Compound No. 2012).

46. A method for the treatment or prevention of diseases, disorders or conditions related to cell adhesion or cell migration mediated by heparan sulfate glycosaminoglycans, comprising the step of administering to a subject in need thereof a therapeutically effective amount of a pharmaceutical composition comprising a compound of the general formula I: 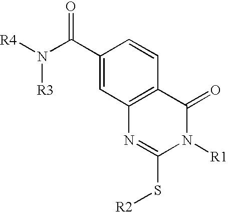
wherein:
R₁ is optionally substituted hydrocarbyl or heterocyclyl;
R₂ is selected from the group consisting of H, (C₁-C₁₂)alkyl, (C₆-C₁₄)aryl-CH₂-, heteroaryl-CH₂-, alkylcarbonyl-CH₂-, (C₆-C ₁₄)arylcarbonyl-CH₂-, and heteroarylcarbonyl-CH₂-;
R₃ and R₄ each is selected from the group consisting of hydrogen, C₁-C₆ alkyl, (C₁-C₆)alkoxy(C₁-C₆)alkyl, and C₁-C₆ alkyl substituted by a group containing a basic nitrogen atom or by a 5-7 membered heterocyclic ring containing one or two heteroatoms, one of them being a basic nitrogen atom, or R₃ and R₄ together with the nitrogen atom to which they are attached form a 5-7 membered saturated heterocyclic ring containing one or two basic nitrogen atoms, optionally substituted on the additional nitrogen atom;
and pharmaceutically acceptable salts thereof.

47-62. (canceled)

63. The method according to claim 46, wherein the compound of formula I is selected from the group consisting of:
2-[[(4-chlorophenyl)methyl]thio]-3-(4-fluorophenyl)-3,4-dihydro -4-oxo-N-[3-(4-morpholinyl)propyl]-7-quinazolinecarboxamide [Compound No. 1];
2-[[(4-methylphenyl)methyl]thio]-3-(4-fluorophenyl)-3,4-dihydro -4-oxo-N-[3-(4-morpholinyl)propyl]-7-quinazolinecarboxamide [Compound No. 2];
2-[[(3-fluorophenyl)methyl]thio]-3-(4-fluorophenyl)-3,4-dihydro -4-oxo-N-[3-(4-morpholinyl)propyl]-7-quinazolinecarboxamide [Compound No. 3];
2-[(2-oxo-2-phenylethyl)thio]-3-(4-fluorophenyl)-3,4-dihydro-4-oxo-N-[3-(4-morpholinyl)propyl]-7-quinazolinecarboxamide [Compound No. 5]; and
2-[[2-[[(3-chlorophenyl)methyl]thio]-3-pentyl-3,4-dihydro-4-oxo -N-(4-methylpiperazinyl)-7-quinazolinecarboxamide [Compound No. 4].

64. The method according to claim 46, wherein the compound of formula I is 2-[[(6-nitro-4H-1,3-benzodioxin -8-yl)methyl]thio]-3-(2-propenyl)-3,4-dihydro-4-oxo -N-[3-(4-morpholinyl)propyl]-7-quinazolinecarboxamide (Compound No. 2010).

65. The method according to claim 46, wherein the compound of formula I is 2-[[(5-acetyl-2-methoxyphenyl) methyl)thio]-3-(phenylmethyl)-3,4-dihydro-4-oxo-N -[3-(1H-imidazol-1-yl)propyl]-7-quinazolinecarboxamide (Compound No. 2011).

66. The method according to claim 46, wherein the compound of formula I is 2-[[(5-acetyl-2-methoxyphenyl) methyl]thio]-3-(phenylmethyl)-3,4-dihydro-4-oxo-N -(1-ethyl-piperidin-4-yl)-7-quinazoline-carboxamide (Compound No. 2012).

67-72. (canceled)

73. A method for modulating the anticoagulant activity of glycosaminoglycans which comprises administering to a subject in need a therapeutically effective amount of a compound of the general formula I in claim 46.

74. The method according to claim 73, wherein the glycosaminoglycan is heparin.

75. The pharmaceutical composition according to claim 1, wherein
R₁ is a hydrocarbyl selected from the group consisting of C₁-C₁₂ alkyl, C₂-C₁₂ alkenyl, C₂-C₁₂ alkynyl, C₃-C₁₀ cycloalkyl, C₃-C₁₀ cycloalkenyl, C₆-C₁₄ aryl, (C₁-C₆)alkyl(C₆-C₁₄)aryl, and (C₆-C₁₄) aryl(C₁-C₁₂)alkyl, or such a hydrocarbyl substituted by at least one radical selected from the group consisting of C₁-C₁₀ alkyl, C₂-C₁₀ alkenyl, C₂-C₁₀ alkynyl, C₇-C₁₂ aralkyl, C₆-C₁₀ aryl, C₇-C₁₂ alkaryl, hydroxy, C₁-C₁₀ alkoxy, C₆-C₁₀ aryloxy, C₁-C₁₀ alkylthio, C₆-C₁₀ arylthio, C₆-C₁₀ arylamino, C₃-C₁₀ cycloalkyl, C₃-C₁₀ cycloalkenyl, amino, C₁-C₁₀ alkylamino, di(C₁-C₁₀)-alkylamino, C₂-C₁₂ alkoxyalkyl, C₂-C₁₂ alkylthioalkyl, C₁-C₁₀ alkylsulfinyl, C₁-C₁₀ alkylsulfonyl, C₆-C₁₀ arylsulfonyl, hydroxy(C₁-C₁₀)alkyl, (C6-C₁₀)aryloxy(C₁-C₁₀)alkyl, (C₁-C₁₀) alkoxycarbonyl, (C6-C₁₀)aryloxycarbonyl, C₂-C₁₁ alkanoyl, (C₇-C₁₁) aroyl, fluoro(C₁-C₁₀)alkyl, oxo, nitro, nitro(C₁-C₁₀)alkyl, cyano, cyano(C₁-C₁₀)alkyl, aminocarbonyl, (C₁-C ₁₀)alkylaminocarbonyl, di(C₁-C₁₀)-alkylaminocarbonyl, aminocarbonyl (C₁-C₁₀)alkyl, aminocarbonyl (C₆-C₁₀) aryl, aminosulfonyl, (C₁-C₁₀)alkylaminosulfonyl, di(C₁-C₁₀)-alkylaminosulfonyl, amidino, carboxy, sulfo, heterocyclyl, and —(CH₂)ₘ-Z-(C₁-C₁₀ alkyl), where m is 1 to 8 and Z is oxygen or sulfur; or R₁ is a heterocyclyl radical derived from a mono-or poly-cyclic ring containing one to three heteroatoms selected from the group consisting of N, O and S;
R₂ is selected from the group consisting of (C₆-C₁₄)aryl-CH ₂-, (C₆-C₁₄)arylcarbonyl-CH₂-, heteroaryl-CH₂- and heteroarylcarbonyl-CH₂-, wherein said aryl or heteroaryl is unsubstituted or substituted by at least one radical selected from the group consisting of C₁-C₁₀ alkyl, C₂-C₁₀ alkenyl, C₂-C₁₀ alkynyl, C₇-C₁₂ aralkyl, C₆-C₁₀ aryl, C₇-C₁₂ alkaryl, hydroxy, C₁-C ₁₀ alkoxy, C₆-C₁₀ aryloxy, C₁-C₁₀ alkylthio, C₆-C₁₀ arylthio, C₆-C ₁₀ arylamino, C₃-C₁₀ cycloalkyl, C₃-C₁₀ cycloalkenyl, amino, C₁-C₁₀ alkylamino, di(C₁-C₁₀)-alkylamino, C₂-C₁₂ alkoxyalkyl, C₂-C₁₂ alkylthioalkyl, C₁-C₁₀ alkylsulfinyl, C₁-C₁₀ alkylsulfonyl, C₆-C₁₀ arylsulfonyl, hydroxy(C₁-C₁₀)alkyl, (C₆-C₁₀)aryloxy(C₁-C₁₀)alkyl, (C₁-C₁₀)alkoxycarbonyl, (C₆-C₁₀)aryloxycarbonyl, C₂-C₁₁ alkanoyl, (C₇-C₁₁)aroyl, fluoro(C₁-C₁₀)alkyl, oxo, nitro, nitro(C₁-C₁₀ )alkyl, cyano, cyano(C₁-C₁₀)alkyl, aminocarbonyl, (C₁-C ₁₀) alkylaminocarbonyl, di(C₁-C₁₀)-alkylaminocarbonyl, aminocarbonyl (C₁-C₁₀) alkyl, aminocarbonyl (C₆-C₁₀) aryl, aminosulfonyl, (C₁-C₁₀)alkylaminosulfonyl, di(C₁-C₁₀)-alkylaminosulfonyl, amidino, carboxy, sulfo, heterocyclyl, and —(CH₂)ₘ-Z-(C₁-C₁₀ alkyl), where m is 1 to 8 and Z is oxygen or sulfur; and wherein said heteroaryl is selected from the group consisting of pyrrolyl, furyl, thienyl, pyrazolyl, imidazolyl, oxazolyl, isoxazolyl thiazolyl, isothiazolyl, pyridyl, 1,3-benzodioxanyl, pyrazinyl, pyrimidinyl, 1,3,4-triazinyl, 1,2,3-triazinyl, 1,3,5-triazinyl, thiazinyl, quinolinyl, isoquinolinyl, benzofuryl, isobenzofuryl, indolyl, imidazo[1,2-a]pyridyl, pyrido[1,2-a]pyrimidinyl, benzimidazolyl, benzthiazolyl, and benzoxazolyl;
R₃ is hydrogen; and
R₄ is selected from the groups consisting of:
(i) (C₁-C₆)alkoxy(C₁-C₆)alkyl;
(ii) (C₁-C₆)alkyl substituted by a group selected from the group consisting of an amino group —NR₅R₆, an ammonium group —N⁺(R₅R₆R₇), a hydrazine group —NR₅—NR₆R7, a hydrazonium group —NR ₅—N⁺(R₆R₇R₈), an ammoniumoxy group —O—N⁺(R₅R₆), an imine group —C═NR₅R₆, an iminium group —C═N⁺(R₅R₆R₇), a guanidine group —NR ₅—C(═NH)—NR₆R₇, and a guanidinium group —NR₅—C(═NH)—N ⁺(R₆R₇R₈), wherein each of R₅, R₆, R₇ and R₈ is H, or optionally substituted C₁-C₁₀ alkyl or C₆-C₁₀ aryl; and
(iii) (C₁-C₆)alkyl substituted by a 5-7 membered heterocyclic ring selected from the group consisting of pyrrolidine, pyrroline, pyrrol, imidazolidine, imidazoline, imidazole, piperidine, dihydropyridine, tetrahydropyridine, pyridine, 1,2-pyrazine, tetrahydropyrimidine, dihydropyrimidine, pyrimidine, 1,4-pyrazine, 1,4-tetrahydropyrazine, 1,4-dihydropyrazine, piperazine, diazepine, oxazolidine, oxazoline, oxazole, morpholino, 1,4-dihydrooxazine, 1,4-oxazine. thiazolidine, thiazoline, thiaazole, thiomorpholino, 1,4-dihydrothiazine, and 1,4-thiazine; or
R₃ and R₄ together with the nitrogen atom to which they are attached form a 5-7 membered saturated heterocyclic ring containing one or two basic nitrogen atoms selected from the group consisting of pyrrolidine, imidazolidine, piperidine, piperazine, and piperazine substituted at the additional nitrogen atom by C₁-C₆ alkyl or by C₁-C₆ alkyl substituted by halo, hydroxy, C₁-C₆ alkoxy or C₆-C₁₀ aryl, or C₂-C₇ alkoxycarbonyl.

76. The pharmaceutical composition according to claim 75, wherein R₁ is selected from the group consisting of pentyl, allyl, phenyl, 4-fluorophenyl, benzyl, 2-furylmethyl and (tetrahydro-2-furyl)methyl;
R₂ is selected from the group consisting of phenyl-CH₂-, 4-methylphenyl -CH₂-, 3-fluorophenyl-CH₂-, 4-fluorophenyl-CH₂-, 3-chlorophenyl -CH₂-, 4-chlorophenyl-CH₂-, phenylcarbonyl-CH₂-, 4-fluoro -phenylcarbonyl-CH₂-, 4-chloro-phenylcarbonyl-CH₂-, 4-pyridyl -CH₂- and 4-oxo-4H-pyrido[1,2-a]pyrimidin-yl-CH₂-;
R₃ is hydrogen; and
R₄is selected from the group consisting of 2-methoxyethyl, 3-(4-morpholinyl)propyl and 3-(1-piperidinyl)propyl; or
R₃ and R₄ together with the nitrogen atom to which they are attached form 4-methylpiperazinyl or 1-piperazinyl-4-carboxylic acid ethyl ester.

77. The pharmaceutical composition according to claim 22, wherein said inflammatory or autoimmune disease, disorder or condition is selected from the group consisting of sepsis, wound associated sepsis, post-septic shock, ischemic leukocyte-mediated tissue damage (ischemia-reperfusion injury) such as myocardial ischemia, platelate-mediated pathologies such as atherosclerosis and clotting, cardiomyopathic disease, stroke, restenosis, infectious meningitis, encephalitis, allergic conjunctivitis, organ/tissue transplant rejection (such as skin, kidney, heart, lung, liver, bone marrow, cornea, pancreas, small bowel, autologous bone marrow transplantation), “graft versus host” disease (GVHD), lupus, frost-bite injury or shock, acute leukocyte-mediated lung injury (such as adult respiratory distress syndrome), asthma, allergic rhinitis, acute pancreatitis, traumatic shock, traumatic brain injury, acute and chronic inflammation such as atopic dermatitis, psoriasis, contact dermal hypersensitivity and inflammatory bowel disease such as Crohn's disease and ulcerative colitis, rheumatoid arthritis, retinitis; multiple sclerosis; amyloid disorders such as Alzheimer's disease and type II diabetes; angiogenesis pathologies selected from the group consisting of tumor angiogenesis, ophthalmologic disorders such as neovascular glaucoma, diabetic retinopathy, macular degeneration (particularly age-related macular degeneration) and uveitis, reperfusion of gastric ulcer, contraception and inducing abortion at early stages of pregnancy; viral disorders selected from the group consisting of hepatitis C, hepatitis B, influenza, rhinovirus infections, cytomegalovirus infections, AIDS and respiratory syncytial virus infections; bone degradation, osteoporosis, osteoarthritis, kidney disease including glomerulonephritis and nephritis; and bacterial infections.

78. The pharmaceutical composition according to claim 22, wherein said disease is cancer or tumor metastasis.

79. The pharmaceutical composition according to claim 31, wherein said selectin is L-selectin.

80. The method according to claim 46, wherein
R₁ a is hydrocarbyl selected from the group consisting of C₁-C₁₂ alkyl, C₂-C₁₂ alkenyl, C₂-C₁₂ alkynyl, C₃-C₁₀ cycloalkyl, C₃-C₁₀ cycloalkenyl, C₆-C₁₄ aryl, (C₁-C₆)alkyl(C₆-C₁₄)aryl, and (C₆-C₁₄) aryl(C₁-C₁₂)alkyl, or such a hydrocarbyl substituted by at least one radical selected from the group consisting of C₁-C₁₀ alkyl, C₂-C₁₀ alkenyl, C₂-C₁₀ alkynyl, C₇-C₁₂ aralkyl, C₆-C₁₀ aryl, C₇-C₁₂ alkaryl, hydroxy, C₁-C₁₀ alkoxy, C₆-C₁₀ aryloxy, C₁-C₁₀ alkylthio, C₆-C₁₀ arylthio, C₆-C₁₀ arylamino, C₃-C₁₀ cycloalkyl, C₃-C₁₀ cycloalkenyl, amino, C₁-C₁₀ alkylamino, di(C₁-C₁₀)-alkylamino, C₂-C₁₂ alkoxyalkyl, C₂-C₁₂ alkylthioalkyl, C1-C₁₀ alkylsulfinyl, C₁-C₁₀ alkylsulfonyl, C₆-C₁₀ arylsulfonyl, hydroxy(C₁-C₁₀)alkyl, (C6-C₁₀)aryloxy(C₁-C₁₀)alkyl, (C₁-C₁₀) alkoxycarbonyl, (C6-C₁₀)aryloxycarbonyl, C₂-C₁₁ alkanoyl, (C₇-C₁₁) aroyl, fluoro(C₁-C₁₀)alkyl, oxo, nitro, nitro(C₁-C₁₀)alkyl, cyano, cyano(C₁-C₁₀)alkyl, aminocarbonyl, (C₁-C ₁₀)alkylaminocarbonyl, di(C₁-C₁₀)-alkylaminocarbonyl, aminocarbonyl(C₁-C₁₀)alkyl, aminocarbonyl(C6-C₁₀)aryl, aminosulfonyl, (C₁-C₁₀)alkylaminosulfonyl, di(C₁-C₁₀)-alkylaminosulfonyl, amidino, carboxy, sulfo, heterocyclyl, and —(CH2)ₘ-Z-(C₁-C₁₀ alkyl), where m is 1 to 8 and Z is oxygen or sulfur; or R₁ is a heterocyclyl radical derived from a mono-or poly-cyclic ring containing one to three heteroatoms selected from the group consisting of N, O and S;
R₂ is selected from the group consisting of (C₆-C₁₄)aryl-CH ₂-, (C₆-C₁₄)arylcarbonyl-CH₂-, heteroaryl-CH₂- and heteroarylcarbonyl-CH₂-, wherein said aryl or heteroaryl is unsubstituted or substituted by at least one radical selected from the group consisting of C₁-C₁₀ alkyl, C₂-C₁₀ alkenyl, C₂-C₁₀ alkynyl, C₇-C₁₂ aralkyl, C₆-C₁₀ aryl, C₇-C₁₂ alkaryl, hydroxy, C₁-C ₁₀ alkoxy, C₆-C₁₀ aryloxy, C₁-C₁₀ alkylthio, C₆-C₁₀ arylthio, C₆-C ₁₀ arylamino, C₃-C₁₀ cycloalkyl, C₃-C₁₀ cycloalkenyl, amino, C₁-C₁₀ alkylamino, di(C₁-C₁₀)-alkylamino, C₂-C₁₂ alkoxyalkyl, C₂-C₁₂ alkylthioalkyl, C₁-C₁₀ alkylsulfinyl, C₁-C₁₀ alkylsulfonyl, C6-C₁₀ arylsulfonyl, hydroxy(C₁-C₁₀)alkyl, (C₆-C₁₀)aryloxy(C₁-C₁₀)alkyl, (C₁-C₁₀)alkoxycarbonyl, (C₆-C₁₀)aryloxycarbonyl, C₂-C₁₁ alkanoyl, (C₇-C₁₁)aroyl, fluoro(C₁-C₁₀)alkyl, oxo, nitro, nitro(C₁-C ₁₀)alkyl, cyano, cyano(C₁-C₁₀)alkyl, aminocarbonyl, (C₁-C ₁₀)alkylaminocarbonyl, di(C₁-C₁₀)-alkylaminocarbonyl, aminocarbonyl (C₁-C₁₀) alkyl, aminocarbonyl (C₆-C₁₀)aryl, aminosulfonyl, (C₁-C₁₀)alkylaminosulfonyl, di(C₁-C₁₀)-alkylaminosulfonyl, amidino, carboxy, sulfo, heterocyclyl, and —(CH₂)ₘ-Z-(C₁-C₁₀ alkyl), where m is 1 to 8 and Z is oxygen or sulfur; and wherein said heteroaryl is selected from the group consisting of pyrrolyl, furyl, thienyl, pyrazolyl, imidazolyl, oxazolyl, isoxazolyl thiazolyl, isothiazolyl, pyridyl, 1,3-benzodioxanyl, pyrazinyl, pyrimidinyl, 1,3,4-triazinyl, 1,2,3-triazinyl, 1,3,5-triazinyl, thiazinyl, quinolinyl, isoquinolinyl, benzofuryl, isobenzofuryl, indolyl, imidazo[1,2-a]pyridyl, pyrido[1,2-a]pyrimidinyl, benzimidazolyl, benzthiazolyl, and benzoxazolyl;
R₃ is hydrogen; and
R₄ is selected from the groups consisting of:
(i) (C₁-C₆)alkoxy(C₁-C₆)alkyl;
(ii) (C₁-C₆)alkyl substituted by a group selected from the group consisting of an amino group —NR₅R₆, an ammonium group —N ⁺(R₅R₆R₇), a hydrazine group —NR₅—NR₆R₇, a hydrazonium group —NR ₅—N⁺(R₆R₇R₈), an ammoniumoxy group —O—N⁺(R₅R₆), an imine group —C═NR₅R₆, an iminium group —C═N⁺(R₅R₆R₇), a guanidine group —NR ₅—C(═NH)—NR₆R₇, and a guanidinium group —NR₅—C(═NH)—N ⁺(R₆R₇R₈), wherein each of R₅, R₆, R₇ and R₈ is H, or optionally substituted C₁-C₁₀ alkyl or C₆-C₁₀ aryl; and
(iii) (C₁-C₆)alkyl substituted by a 5-7 membered heterocyclic ring selected from the group consisting of pyrrolidine, pyrroline, pyrrol, imidazolidine, imidazoline, imidazole, piperidine, dihydropyridine, tetrahydropyridine, pyridine, 1,2-pyrazine, tetrahydropyrimidine, dihydropyrimidine, pyrimidine, 1,4-pyrazine, 1,4-tetrahydropyrazine, 1,4-dihydropyrazine, piperazine, diazepine, oxazolidine, oxazoline, oxazole, morpholino, 1,4-dihydrooxazine, 1,4-oxazine. thiazolidine, thiazoline, thiaazole, thiomorpholino, 1,4-dihydrothiazine, and 1,4-thiazine; or
R₃ and R₄ together with the nitrogen atom to which they are attached form a 5-7 membered saturated heterocyclic ring containing one or two basic nitrogen atoms selected from the group consisting of pyrrolidine, imidazolidine, piperidine, piperazine, and piperazine substituted at the additional nitrogen atom by C₁-C₆ alkyl or by C₁-C₆ alkyl substituted by halo, hydroxy, C₁-C₆ alkoxy or C₆-C₁₀ aryl, or C₂-C₇ alkoxycarbonyl.

81. The method according to claim 80, wherein R₁ is selected from the group consisting of pentyl, allyl, phenyl, 4-fluorophenyl, benzyl, 2-furylmethyl and (tetrahydro-2-furyl)methyl;
R₂ is selected from the group consisting of phenyl-CH₂-, 4-methylphenyl -CH₂-, 3-fluorophenyl-CH₂-, 4-fluorophenyl-CH₂-, 3-chlorophenyl -CH₂-, 4-chlorophenyl-CH₂-, phenylcarbonyl-CH₂-, 4-fluoro -phenylcarbonyl-CH₂-, 4-chloro-phenylcarbonyl-CH₂-, 4-pyridyl -CH₂- and 4-oxo-4H-pyrido[1,2-a]pyrimidin-yl-CH₂-;
R₃ is hydrogen; and
R₄is selected from the group consisting of 2-methoxyethyl, 3-(4-morpholinyl)propyl and 3-(1-piperidinyl)propyl; or
R₃ and R₄ together with the nitrogen atom to which they are attached form 4-methylpiperazinyl or 1-piperazinyl-4-carboxylic acid ethyl ester.

82. The method according to claim 46, for the treatment or prevention of inflammatory or autoimmune diseases, disorders or conditions related to cell adhesion or cell migration mediated by heparan sulfate glycosaminoglycans.

83. The method according to claim 82, wherein said inflammatory or autoimmune disease, disorder or condition is selected from the group consisting of sepsis, wound associated sepsis, post-septic shock, ischemic leukocyte-mediated tissue damage (ischemia-reperfusion injury) such as myocardial ischemia, platelate-mediated pathologies such as atherosclerosis and clotting, cardiomyopathic disease, stroke, restenosis, infectious meningitis, encephalitis, allergic conjunctivitis, organ/tissue transplant rejection (such as skin, kidney, heart, lung, liver, bone marrow, cornea, pancreas, small bowel, autologous bone marrow transplantation), “graft versus host” disease (GVHD), lupus, frost-bite injury or shock, acute leukocyte-mediated lung injury (such as adult respiratory distress syndrome), asthma, allergic rhinitis, acute pancreatitis, traumatic shock, traumatic brain injury, acute and chronic inflammation such as atopic dermatitis, psoriasis, contact dermal hypersensitivity and inflammatory bowel disease such as Crohn's disease and ulcerative colitis, rheumatoid arthritis, retinitis; multiple sclerosis; amyloid disorders such as Alzheimer's disease and type II diabetes; angiogenesis pathologies selected from the group consisting of tumor angiogenesis, ophthalmologic disorders such as neovascular glaucoma, diabetic retinopathy, macular degeneration (particularly age-related macular degeneration) and uveitis, reperfusion of gastric ulcer, contraception and inducing abortion at early stages of pregnancy; viral disorders selected from the group consisting of hepatitis C, hepatitis B, influenza, rhinovirus infections, cytomegalovirus infections, AIDS and respiratory syncytial virus infections; bone degradation, osteoporosis, osteoarthritis, kidney disease including glomerulonephritis and nephritis; and bacterial infections.

84. The method according to claim 82, wherein said disease is cancer or tumor metastasis.
